(19) United States
(12) Patent Application Publication
(10) Pub. No.: US 2017/0007620 A1
(43) Pub. Date: Jan. 12, 2017

(54) Method of replenishing steroid hormones in neutered mammals comprising administration of dehydroepiandrosterone (DHEA) and specific metabolites in order to increase steroid hormone levels from those of neutered mammals associated with high cancer risk, to steroid hormone levels of gonadally-intact mammals associated with reduced cancer risk, without inducing the side effects that would occur without metabolite supplementation.

(71) Applicant: Jonathan Wesley Nyce, Lansdale, PA (US)

(72) Inventor: Jonathan Wesley Nyce, Lansdale, PA (US)

(21) Appl. No.: 14/792,654

(22) Filed: Jul. 7, 2015

(51) Int. Cl. A61K 31/566; A61K 33/00; A61K 31/355; A61K 31/122; A61K 31/197; A61K 31/405; A61K 31/675; A61K 31/198; A61K 31/7076; A61K 31/375; A61K 31/519; A61K 31/09

(52) U.S. Cl. CPC A61K 31/566; A61K 31/519; A61K 33/00; A61K 31/355; A61K 31/122; A61K 31/09; A61K 31/405; A61K 31/675; A61K 31/198; A61K 31/7076; A61K 31/375; A61K 31/197

(57) ABSTRACT

The low steroid hormone levels of neutered or aging mammals is associated with a dramatic increase in risk of cancer. This invention pertains to a natural method of replenishing the low steroid hormone levels of neutered or aging mammals, levels that are associated with high cancer risk, to levels of those of non-neutered or young animals, levels that are associated with a low cancer risk. The invention also pertains to methods of maintaining healthy levels of several important natural metabolites that can become depleted during the steroid hormone replenishment process.

### STATEMENT REGARDING FEDERALLY SPONSORED RESEARCH OR DEVELOPMENT

Although the applicant has received several federal research grants in the past (e.g., R01 CA47217; R29 CA47217), the work underlying this invention is unrelated to the work conducted under such grants.

A method of replenishing steroid hormones in neutered mammals comprising administration of dehydroepiandrosterone (DHEA) and specific metabolites in order to increase steroid hormone levels from those of neutered mammals associated with high cancer risk, to steroid hormone levels of gonadally-intact mammals associated with reduced cancer risk, without inducing the side effects that would occur without metabolite supplementation.

### CROSS REFERENCE TO RELATED PATENTS

None

### INVENTOR

Dr. Jonathan Nyce, Canine Advanced Genetic Testing, LLC, 399 Arcola Road, Collegeville, Pa. 19426. Home address: 2267 Warner Road, Lansdale, Pa. 19446

### SEQUENCE LISTING

Not Applicable

### PRIOR DISCLOSURE(S)

There have been no prior disclosures of this invention

### BACKGROUND OF THE INVENTION

1. Field of the Invention

The field is directed to compositions and methods related to a novel treatment of neutered and/or aging mammals by removing said neutered/aging mammals from a state of negligible steroid hormone levels associated with an increased risk of cancer, to a state of reconstituted hormone levels typical of non-neutered and/or young animals, which levels in non-neutered or young mammals are associated with reduced cancer risk. The agent employed to effect this treatment is Dehydroepiandrosterone (DHEA), its sulfated derivative Dehydroepiandrosterone-sulfate (DHEAS), and/or their derivatives, metabolites and precursors, administered at doses sufficient to induce Extra-Gonadal Steroid hormone Synthesis (EGSS). The invention also relates to a method to maintain sufficient levels of the isoprenoid N6-isopentenyladenosine (IPA) and/or the essential enzymatic cofactor tetrahydrobiopterin (BH4), thereby preventing the auto-inflammatory and other negative side effects of DHEA-mediated EGSS. A final embodiment of the invention is the addition of potassium nitrate or other nitric oxide donor to the EGSS formulation to prevent nitric oxide depletion, one or more tocotrienols (alpha, beta, gamma, delta) and/or ubiquinone to mitigate ubiquinone depletion, monoamine precursors and cofactors to prevent monoamine depletion, and folinic acid (or metabolites of the folate pathway) to replenish folate metabolites diminished during DHEA-mediated EGSS.

2. Description of Related Art

The steroid hormones play vital roles in mammalian physiology. (See Salerni, S. et al, Eur J Clin Invest. 2015 Apr. 3; Sathish V, Martin Y N, Prakash Y S, Pharmacol Ther. 2015 Jan. 14; Chamouni A, Oury F, Arch Biochem Biophys. 2014 Nov. 1; 561:147-53; Oury F, Ann N Y Acad Sci. 2012 July; 1260:1-7; Callewaert F, Boonen S, Vanderschueren D. Trends Endocrinol Metab. 2010 February; 21(2):89-95). All mammals experience a decline in steroid hormone levels with increasing age. (See Rohrmann, Sabine et al, Clin Endocrinol (Oxf) 2011 August, 75(2):232-239; Sorwell, K G and Urbanski, H F, J. Neuroendocrin 2013, 25(11):1062-1069, November 2013). There are many known associations between decreasing hormone levels and increasing risk for a wide array of common diseases. (Cunningham G R, Asian J Androl. 2015 March-April; 17(2):192-6; Srinath, R et al, J Clin Endocrinol Metab. 2015 April; 100(4):1602-8; Basualto-Alarcón C, et al, Front Endocrinol (Lausanne). 2014 Dec. 18; 5:217; Morgentaler A, Asian J Androl. 2015 January-February; 17(1):26-31). Although steroid hormone levels gradually decline with age in intact animals, such decline is catastrophic in animals that undergo neutering to prevent them from procreating, for behavior modification, or for other reasons.

The ablation of hormonal levels consequent to such neutering is associated with a dramatically increased risk of cancer and other illnesses. For example, in a study of 759 client owned Golden Retrievers, Torres de la Riva et al showed that 10% of neutered male dogs showed hip dysplasia, twice the number observed in intact dogs; there were no cases of cranial cruciate ligament tear (CCL) in intact dogs, versus 5% of male and 8% of female neutered Retrievers developing CCL; almost 10% of neutered male Retrievers developed lymphosarcoma, three times more than in intact males (See also Villamil, J A, et. al., J Cancer Epidemiol 2009:591753); hemangiosarcoma was four times more prevalent in neutered female Retrievers than in their intact counterparts; and while there were no cases of mast cell cancer in intact Female Retrievers, nearly 6% of neutered females developed this fatal cancer. (Torres de la Riva, G et al, PLoS One, 2013, 8(2):e55937). Several studies showed that neutered male dogs are four times more likely to develop prostate cancer than intact dogs (Teske E, Naan E C, van Dijk E M, van Garderen E, Schalken J A. Mol Cell Endocrinol. 2002 Nov. 29; 197(1-2):251-255; Sorenmo K U, Goldschmidt M, Shofer F, Ferrocone J. Vet Comparative Oncology.2003 March; 1 (1): 48). Clearly, the ablation of the hormonal system caused by neutering produces dramatic increases in risk of the major diseases affecting domesticated dogs. Similar effects are observed in neutered cats (Rissetto, K et al, J Am Anim Hosp Assoc. 2011 January-February; 47(1):28-36), and even in aging human beings (Rohrmann, S., et. al., Clin Endocrinol (Oxf). 2011 August; 75(2): 232-239. doi: 10.1111/j.1365-2265.2011.04043.x). A method of restoring the protective hormonal system in neutered dogs and cats would thus be of immense importance to pets and their owners.

One possible way to effect replacement of missing hormones in neutered animals would be to administer sex steroids such as testosterone or estradiol. But this type of hormone replacement focuses too far upstream and therefore does not include all hormonal factors missing in neutered dogs, some of which appear to play a direct role in cancer prevention (see below). Furthermore, direct treatment with testosterone or estradiol bypasses homeostatic mechanisms designed to precisely adjust steroid hormone synthesis to physiological need. A precursor to steroid hormone synthesis that would be subject to such physiological control would be much preferred.

Dehydroepiandrosterone (DHEA; CAS 53-43-0) and its sulfated metabolite, Dehydroepiandrosterone sulfate (DHEAS; CAS 651-48-9) is a steroid hormone precursor, and is found in the highest levels of any steroid in humans and higher primates. Because no receptor for it has been found, DHEA itself does not fit the definition of a hormone (See Widstrom, Richard L. Seminars in Reproductive Medicine 12/2004; 22(4):289-98; Labrie, F et. al. Journal of Endocrinology (2005) 187, 169-196). It is frequently noted that the decrease in serum DHEA levels with age models closely the increase in cancer risk in mammalian species. Low serum DHEA levels correlate with increased risk of cancer, cardiovascular disease, asthma, atopic dermatitis, and other illnesses. Many laboratories, including the author's (Nyce J W et al, Carcinogenesis. 1984 January; 5(1):57-62), have demonstrated that DHEA is capable of inhibiting carcinogenesis in a large number of animal models (For a review see Williams, John, Lipids, 04/2000; 35(3):325-31).

In humans, almost all DHEA is synthesized in the adrenal glands, with a small amount synthesized in gonadal tissues (See Dillon, J S, Curr Drug Targets Inflamm Allergy. 2005 June; 4(3):377-85). This is the reverse of what is observed in animals such as rats, mice, dogs, cats and guinea pigs where the secretion of DHEA and sex steroids takes place in the gonads (See Bélanger B. et. al., Journal of Steroid Biochemistry 32 695-698, 1989; Labrie F, Dupont A & Bélanger A. In Important Advances in Oncology, pp 193-217,1985. Eds V T de Vita, S Hellman & S A; Rosenberg. Philadelphia: J. B. Lippincott; Labrie F, Belanger A, Cusan L & Candas B, Journal of Clinical Endocrinology and Metabolism 82:2403-2409). van der Molen, H J, et al. Biochim Biophys Acta, 1971 Nov. 5; 248(2):343-62; Odell W D, Parker L N, Endocr Res. 1984-1985; 10(3-4):617-30). Thus, in dogs, cats and most other non-primate mammals, there is extremely little or no DHEA synthesis in the adrenal glands, with all or almost all synthesis occurring in gonadal tissues.(ibid; Mongillo P et al, Res Vet Sci. 2014 February; 96(1):33-8). Cutler G B Jr, et al., Endocrinology. 1978 December; 103(6):2112-8; Ahlem C N, et. al., Steroids. 2011 June; 76(7):669-74). Therefore, following castration, serum levels of DHEA and the sex steroids drop to virtually undetectable levels. It has been recently shown that the enzymes that could synthesize sex steroids exist in extragonadal tissues. (Labrie, F et al, Front Neuroendocrinol. 2001 July; 22(3):185-212; Aizawa K et al, Am J Physiol Endocrinol Metab. 2007 February; 292(2):E577-84. Epub 2006 Oct. 3. Inoue, Takayoshi et al, Mol Cell Endocrinol 2012 Oct. 22; 362(1-2):19-28. Epub 2012 May 22). The extragonadal synthesis of steroid hormones (EGSS) is eliminated in neutered dogs, cats, etc. due to the fact that, since DHEA in these species is synthesized almost entirely in the gonads and not the adrenal glands, there is no steroid precursor to enable EGSS to occur.

DHEA/DHEAS has been proposed as a treatment or prevention in humans for a variety of diseases including vaginal atrophy, hypogonadism, diminished libido, osteoporosis, urinary incontinence, ovarian cancer, uterine cancer, skin atrophy, for contraception, and, in combination with an estradiol and/or progestin, for the treatment of menopause (Labrie, F., European Patent EP0680327); depression (Michael, Herbert, WIPO Patent Application WO/1996/025164); Systemic Lupus Erythematosus (McGuire, James L., U.S. Pat. No. 5,567,696); and Primary Adrenal Deficiency and Addison's Disease (Yen, Samuel S. C. and Berger, Brian, WIPO Patent Application WO/1998/032445). The author of the instant patent has several issued patents on the use of DHEA in the treatment of cancer, asthma and other diseases. (Nyce, Jonathan W., U.S. Pat. No. 7,893,044; Nyce, Jonathan W., U.S. Pat. No. 5,527,789; European Patent EP0627921; Nyce, Jonathan W., U.S. Pat. No. 6,087,351; Nyce, Jonathan W., U.S. Pat. No. 6,670,349; Nyce, Jonathan W., U.S. Pat. No. 7,456,161).

The use of DHEA to prevent or treat chronic fatigue syndrome and/or fibromyalgia in humans, dogs and cats has been proposed (Zenk, Ronald, Zenk, John L., WIPO Patent Application WO/2002/043737), but this invention specifically excluded doses of DHEA that could lead to the synthesis of steroid hormones. Also, the treatment or prevention of male or female menopause symptoms using a combination of a sex hormone binding globulin synthesis inhibiting agent and one or more steroids has been reported (Van Der, Hoop Roland Gerritsen, WIPO Patent Application WO/2003/002123), but this application focuses on improvement of libido and/or sexual response.

The use of DHEA to replenish steroid hormone levels in neutered mammals, so as to increase steroid hormone levels to those of gonadally-intact animals, has not heretofore been proposed.

### BRIEF SUMMARY OF THE INVENTION

Our laboratory has made the novel discovery that normal or near normal levels of steroid hormones can be restored in neutered animals by administering DHEA or DHEAS to achieve EGSS. This eliminates the negligible steroid levels of neutered dogs that are associated with dramatic increases in risk for cancer and other diseases as described above, restoring them to the normal or near normal steroid hormone levels of non-neutered animals that are associated with the much decreased cancer risk experienced by intact animals. However, our laboratory has also made the novel discovery that the doses of DHEA that are required to induce EGSS-mediated reconstitution of normal or near normal steroid hormone levels in neutered animals are capable of causing many serious side effects. These side effects include (1) an auto-inflammatory response affecting skin, eyes, and mucous membranes; (2) diminution of neurotransmitters, resulting in neuromuscular defects; (3) a sensitivity to fungal and other infections induced by diminution of nitric oxide synthesis; (4) loss of nitric oxide-mediated vascular control; (5) depletion of ubiquinone and a consequent impairment of mitochondrial respiration; and (6) periodic fever. We discovered that these side effects are caused by DHEA-mediated depletion of IPA, BH4, nitric oxide, products of the folate pathway, monoamines and ubiquinone, which we proved by showing that (1) IPA, BH4, NO, products of the folate pathway (e.g. AMP, ADP, and ATP), and ubiquinone are all significantly depleted during DHEA-mediated EGSS; and (2) the side effects of DHEA-mediated EGSS can be essentially eliminated by co-administration of IPA, BH4, a nitric oxide donor such as potassium nitrate, folinic acid (or folate pathway metabolites), monoamine precursors and cofactors, and either ubiquinone itself, or tocotrienol±ubiquinone to maintain ubiquinone levels. In order to properly illustrate the seriousness of the side effects caused by depletion of these metabolites, we must now discuss their role in intermediary metabolism, and the pathologic sequellae that ensue when they become depleted.

IPA. Our laboratory has made the novel finding that IPA, a product of the mevalonate pathway, becomes depleted when exogenous DHEA is administered in amounts sufficient to induce EGSS. IPA plays several important roles in the cell, and its depletion would be expected to be deleterious on many levels. For example, in the biosynthesis of selenoproteins, the adenosine at residue 37 of tRNA molecules that bind codons starting with UGA (normally a stop signal in mRNA) is modified to create IPA within the tRNA molecule (Bifulco, M. Malfitano, A M, Proto, M C et al. Anticancer Agents Med Chem 2008, 8(2): 2000-2004). The T4 deiodinase that converts inactive T4 to active T3 is such a selenoprotein. There are at least 24 additional mammalian selenoproteins, the identification and investigation of which has been hampered by the fact that the selenocysteine insertion signal is UGA, which heretofore had been considered exclusively a stop codon; hence most selenoproteins were unidentified in genetic databases until newer algorithms were devised to identify their presence. (Kryukov, G. V. et al, J. Biol. Chem. 274, 33888-33897, 1999). IPA depletion not only impairs the synthesis/function of the selenoprotein T4 deiodinase, but may also exert an oncogenic pressure upon the cell, Thus, Spinola et al have shown that the enzyme (TRIT1) that catalyzes the transfer of the isopentenyl moiety to the target tRNA is 6-14 fold down-regulated in lung adenocarcinomas as compared to normal lung tissue (Spinola M et al, Oncogene. 2005 Aug. 18; 24(35):5502-9). This identifies TRIT1 as a tumor suppressor gene. IPA is then identified as a tumor suppressor molecule whose absence in DHEA-treated animals would almost certainly be oncogenic, as it would cause the same diminution of IPA addition to tRNA that TRIT1 down-regulation causes. This shows that, in animals treated with DHEA to induce EGSS, it is critical to replenish IPA to maintain the tumor suppressor activity of TRIT1. The tumor suppressor function of selenoproteins was also demonstrated in an animal model (Hudson, T. S. et al, Carcinogenesis 33(6):1225-1230, 2012). Indeed, pharmacological doses of DHEA have been shown to sometimes cause cancer rather than prevent it (See Hayashi F., Carcinogenesis. 1994 October; 15(10):2215-9). This can now be explained as due to the DHEA-mediated depletion of IPA, with the downstream depletion of selenomethane, leading to the same endpoint—depletion of selenoproteins—as would occur with TRIT1 tumor suppressor gene inactivation.

Selenoproteins, and therefore IPA, have also been shown to play a critical role in inflammation. For example, loss of function of the selenoprotein Sep15 leads to dramatic induction of STAT-1 regulated inflammatory genes. (See Tsuji, P. A. et al, PLoS One, 2015, 10(4):e0124487). Selenoprotein synthesis is a critical determinant of the balanced biosynthesis of pro- and anti-inflammatory oxylipids in macrophages. (See Mattmiller, S A et al, J. Nut Biochem 25(6):647-54, 2014). Auto-inflammatory conditions caused by genetic defects in the mevalonate pathway (in which the isopentenyl moiety is synthesized) have been described (e.g., Caso, Francesco et al., Int J. Rheumatol 2013, Oct. 24; van der Burgh, R et al, Clin Immunol 147(3):197-206, 2013). Aspects of these monogenic auto-inflammatory diseases closely resemble some of the symptoms observed in animals treated with DHEA to induce EGSS. These include fever, inflammation of the eyes, skin and serous membranes.

IPA also regulates Natural Killer (NK) cell activity. NK cells are lymphocytes of the innate immune system that can kill transformed and pathogen-infected cells directly. They also secrete a variety of cytokines and chemokines (e.g., TNF-α, IFN-γ, CCL3, CCL5, IL-8, IL-10, etc) through which they shape the subsequent adaptive immune response. Properly functioning NK cells therefore play critical roles in immune defense and regulation of inflammatory responses. IPA induces expansion and activation of the NK cell compartment. DHEA has been shown to increase NK cell number and cytotoxicity (Khorram, O et al, J Gerontol A Biol Sci Med Sci. 1997 January; 52(1):M1-7). During DHEA-mediated stimulation of EGSS, when DHEA stimulates NK cell activity and NK-mediated cytotoxicity, it is at the same time eliminating IPA and therefore the controls upon NK cell activity and cytotoxicity that IPA provides. NK cells are known to moderate the inflammatory response in the eye. (Liu Q et al, Am J Pathol. 2012 August; 181(2):452-62). We see identical pathology in dogs treated with DHEA to restore steroid hormone levels to normal via EGSS. Evidence that such ocular pathology is caused by IPA depletion comes from the fact that reconstitution of IPA (and BH4) prevents such DHEA-induced ocular inflammation from occurring.

Since we have observed that motor incoordination sometimes occurs in animals undergoing DHEA-mediated EGSS, it is important to note that reduction in neuronal selenoprotein synthesis has been demonstrated to lead to loss of motor coordination in mice. (See Seeher, S. et al, Biochem J. 462(1):67-75, 2014).

It had been thought that the sole source of IPA was the turnover of isopentenylated tRNA. However, studies in yeast (Laten, H M and Zahareas-Doktor, S PNAS USA February 1985; 82(4):1113-1115) and plants (Stot, Crister A, Karel Dolezal, Anders Nordstrom et al. PNAS USA Dec. 19, 2000; 97(26):14778-14783) show that the major source of free IPA is not turnover of isopentenylated tRNA, but rather a separate pathway independent of such turnover. Similar means to synthesize free IPA must also exist in animal tissues, as is supported by several findings. First, statins (anti-cholesterol drugs that inhibit HMG CoA reductase, the rate limiting step in isoprenoid and cholesterol synthesis) inhibit cell growth and division by arresting cells in the G1 phase of the cell cycle. Isopentenyladenine (the free base form of IPA) is approximately 100 fold more active in restoring DNA synthesis in statin-treated cells than is mevalonate, the direct product of HMG Co A reductase (See Huneeus, V Q, Wiley, M H and Siperstein, M D, PNAS USA October 1980: 77(10):5842-5846). Second, we have discovered that addition of IPA can completely prevent the ophthalmic inflammatory reaction that can occur in dogs following administration of DHEA (see below). These findings, along with the studies in yeast and plants noted above, suggest that all organisms possess synthetic pathways to produce free IPA, and that such IPA has important growth regulatory and/or immunoregulatory roles. An alternative way to replenish IPA, rather than administration of IPA itself, is to administer mevalonate, the precursor molecule for all isoprenoid moieties, or geranylgeraniol, geraniol, or farnesol, which are natural isoprenoid donors.

BH4. Our laboratory has also made the novel finding that the pteridine BH4 is depleted in animals undergoing DHEA-mediated EGSS. BH4 is a required cofactor for many critical enzyme systems including four aromatic amino acid hydroxylases, alkylglycerol mono-oxygenase and three NOS (NO synthase) isoenzymes. It thus plays a critical role in monoamine neurotransmitter formation, cardiovascular and endothelial function, the immune response and pain sensitivity. Within the brain, BH4 is absolutely required for the synthesis of the monoamine (MA) neurotransmitters dopamine (DA), norepinephrine, epinephrine (E), and serotonin (5-HT), the novel gaseous neurotransmitter nitric oxide and the production of yet to be fully identified 1-O-alkylglycerol-derived lipids. (See Kapatos, G., The neurobiology of tetrahydrobiopterin biosynthesis, IUBMB Life 65(4):323-33, 2103). Depression (Pan, Let al, Brit Med J Case Rep bcr0320113927, 2011) and hypopigmentation of the skin have also been reported in animals and persons with BH4 deficiency. (Nagasakin, Y. et al., Pediatr Res. 1999 April; 45(4 Pt 1):465-73).

DHEA-mediated BH4 depletion during induction of EGSS can lead to increased levels of tissue phenylalanine, creating a situation mimicking the genetic disease phenylketonuria (PKN). PKN is known to cause symptoms similar to those we have observed in dogs and cats administered DHEA to induce EGSS, including eczema/atopic dermatitis, an increased incidence of pyogenic infections, an increased incidence of keratosis pilaris, scleroderma-like plaques, and hair loss. (See Scriver, C R and Clow, C L, Annu Rev Genet. 1980; 14:179-202). In human PKU caused by mutations in one of several genes involved in BH4 synthesis, including GCH1, PCBD1, PTS, and QDPR, leading to BH4 deficiency, symptoms can be largely eliminated by pharmacological treatment with BH4 or a BH4 precursor molecule (Sanford, Mark; Keating, Gillian M. (2009). “Sapropterin”. Drugs 69 (4): 461-76). Other methods to reduce phenylalanine levels consequent to BH4 deficiency and prevent the negative sequellae of excessive phenylalanine include (1) dietary restriction so as not to ingest foods high in phenylalanine (van Spronsen F J, Enns G M, Mol Genet Metab. 2010; 99 Suppl 1:S90-5), (2) dietary use of casein glycomacropeptide (a milk-derived peptide containing extremely low amounts of phenylalanine (Strisciuglio P and Concolino D, Metabolites. 2014 Nov. 4; 4(4):1007-17. doi: 10.3390/metabo4041007;), and (3) dietary supplementation with large neutral amino acids (LNAAs, e.g. leucine, tyrosine, tryptophan, methionine, histidine, isoleucine, valine, threonine, etc.) which compete with phenylalanine for specific carrier proteins that transport LNAAs across the intestinal mucosa into the blood and across the blood brain barrier into the brain (Ney D M, Blank R D, and Hansen K E, Curr Opin Clin Nutr Metab Care. 2014 January; 17(1):61-8). Each of these techniques is envisioned as additional embodiments of the instant invention to reduce phenylalanine levels in animals depleted of BH4 during DHEA-mediated EGSS, although reconstitution of BH4 by supplementation with BH4 or its prodrug, Sepiaopterin, remains the preferred embodiment for DHEA-mediated BH4 depletion (see below).

Nitric Oxide (NO). BH4 is a necessary cofactor for the enzymatic synthesis of NO, as noted above, and NO is depleted during DHEA-mediated EGSS. However, BH4 reconstitution is insufficient to reconstitute NOS activity because NADPH is also required for NO synthesis, and NADPH is diminished during DHEA-mediated EGSS. However, various NO donors are available that can reconstitute NO non-enzymatically. For example, potassium nitrate (KNO₃, CAS 7757-79-1), a common food preservative, directly breaks down to NO after ingestion, and can replenish NO under clinical conditions in which it has been depleted (See Baliga, R S, et al, Respiratory and Critical Care Medicine, Abstract Issue, B63, Experimental models in pulmonary hypertension, American Thoracic Society International Conference Abstracts, 2012). We have made the novel observation that potassium nitrate can replenish NO in animals undergoing DHEA-mediated EGSS, eliminating the symptoms associated with NO depletion.

NO is important as a toxic defense molecule against infectious organisms. It also regulates the functional activity, growth and death of many immune and inflammatory cell types including macrophages, T lymphocytes, antigen-presenting cells, mast cells, neutrophils and natural killer cells.(See Coleman, J W, Int Immunopharmacol. 2001 August; 1(8):1397-406). Increased susceptibility to infections occurs when NO is limiting (See Olekhnovitch, R et al, J. Clin Invest 124(4):1711-1722, 2014). As noted above, susceptibility to infections, particularly in the ear and eye, have been noted in dogs and cats treated with DHEA in amounts sufficient to induce EGSS. Again, such susceptibility to infections is eliminated when DHEA treatment includes concurrent BH4 (potassium nitrate and IPA; see below).

Some vascular effects, primarily an induction of vasoconstriction, have been observed during DHEA-mediated EGSS, and these appear to be entirely due to NO depletion since they can be prevented by co-administration of NO donors as noted above. NO was originally described as endothelial relaxing factor, and when it becomes depleted, blood vessels become constricted, increasing blood pressure, decreasing blood flow, and leading to fluid retention in limbs (Sharma, R and Davidoff, M N, Oxidative stress and endothelial dysfunction in heart failure, Congest Heart Fail. 2002 May-June; 8(3):165-72). Fluid retention subsequent to NO depletion has been observed in dogs treated with DHEA in amounts sufficient to induce EGSS. Such symptoms are eliminated when treatment includes concurrent BH4, potassium nitrate and IPA.

Ubiquinone depletion. Ubiquinone (CAS Number 606-06-4; Coenzyme Q10) is a critical component of the mitochondrial respiratory chain, participating in electron transport in NADH-coenzyme Q reductase (complex I), succinate coenzyme Q reductase (complex II) and the cytochrome system. (See Nakamaru-Ogiso E et. al., J Bioenerg Biomembr. 2014, 46(4):269-77). It is especially important with respect to the function of muscle tissue in organs with high energy expenditure, for example in the heart. (Khorrami A, et. al., Drug Res (Stuttg). 2014 April; 64(4):177-81). Over the short term, ubiquinone levels are increased upon DHEA exposure. However, after continuous treatment with DHEA at doses capable of inducing EGSS, ubiquinone levels decline. (FIG. 3). We have found that supplementation with ubiquinone and tocotrienols maintains normal or near normal ubiquinone levels in animals treated with DHEA to induce EGSS (see below). Supplementation with the combination of ubiquinone and tocotrienols appears to be more effective at retaining normal or near normal ubiquinone levels than does supplementation with ubiquinone alone. Bentinger, M et. al. (Biofactors, 2008; 32 (1-4):99-111) have recently reported that tocotrienols stimulate the synthesis of endogenous ubiquinone, and it is possible that this effect is responsible for the dramatically improved reconstitution when ubiquinone plus tocotrienols were used in our experiments.

Depletion of folate pathway intermediates. DHEA-mediated EGSS depresses NADPH production via its inhibition of G6PD. Therefore, pathways that depend heavily on NADPH are inhibited during DHEA-mediated EGSS. The one carbon pool (folate) pathway is one such pathway. We have discovered that one carbon pool products (e.g., purines, pyrimidines, S-adenosylmethionine) are profoundly depleted during DHEA-mediated EGSS. This is similar to the effect of methotrexate (CAS 59-05-2), a cancer chemotherapy agent which inhibits the rate limiting enzyme of the one carbon pool pathway, Dihydrofolate Reductase (CAS CAS9002-03-3; See Schalinskel, K L and Steele, R D Carcinogenesis vol. 17 no.8 pp.1695-1700, 1996). Methotrexate is often administered to cancer patients at doses high enough to be lethal to the patient, but with folinic acid “rescue” to reconstitute one carbon pool metabolism in a timely enough fashion to prevent such lethality (See Borsi, J D et al, Pediatr Hematol Oncol. 1990; 7(4):347-63; folinic acid CAS 1492-18-8). While one carbon pool disruption during DHEA-mediated EGSS can have very positive effects with respect to reduction of the adenosine levels in tumor microenvironments, adenosine levels which contribute strongly to the inactivation of the host immune system (via activation of Adenosine A2A receptors on host immune cells; see Leone R D, Lo Y C, and Powell J D, Comput Struct Biotechnol J. 2015 Apr. 8;13:265-72. doi: 10.1016/j.csbj.2015.03.008. eCollection 2015), it can also cause a slowly progressive polyneuropathy with predominant involvement of the lower extremities (See Koike, H. et al Neurology. 84(10):1026-33, 2015). We have observed similar lower extremity neuropathy in animals undergoing DHEA-mediated EGSS without supplementation, and prevention of such neuropathy with fully supplemented treatment including folinic acid (see below). An optional way to replenish critical metabolites depleted during DHEA-mediated EGSS is to administer purines (e.g., adenine, guanine or hypoxanthine, their nucleosides or nucleotides) and a pyrimidine (e.g., uracil, its nucleoside or nucleotides), along with S-adenosylmethionine (SAMe). See FIG. 4.

Depletion of monoamines. A variety of monoamines are critical for normal physiology. These include serotonin (CAS 50-67-9), dopamine (CAS 51-61-6), melatonin (CAS 73-31-4), epinephrine (CAS 51-43-4) and norepinephrine (CAS 51-41-2). (See, for example, Perrier, J F and Cotel, F, Curr Opin Neurobiol. 2014 Dec. 29; 33C:1-7. doi: 10.1016/j.conb.2014.12.008). As illustrated in FIG. 7, depletion of BH4 suppresses synthesis of L-DOPA, from which dopamine, norepinephrine and epinephrine are subsequently synthesized. Replenishment of BH4 in DHEA-treated animals relieves this suppression, restoring normal or near normal dopamine, norepinephrine and epinephrine levels. However, a cost effective alternative to BH4 supplementation to relieve suppression of this pathway in DHEA-treated animals is to administer a reconstitution protocol that includes L-DOPA (CAS 59-92-7), Pyridoxine (Vitamin B6; pyridoxal 5′-phosphate; CAS 65-23-6), ascorbate (Vitamin C, CAS 50-81-7), and S-Adenosyl-methionine (SAMe; CAS 29908-03-0), albeit without alleviating phenylalanine build up, as BH4 supplementation does. However, this alternative protocol can lead to emesis in some dogs due to L-DOPA's systemic conversion to dopamine. Carbidopa (See Lotti, V J and Clark, C, Eur J Pharmacol. 1974 March; 25(3):322-5), domperidone (See Shuto, K et. al. (J Pharmacobiodyn. 1980 December; 3(12):709-14), and other dopamine antagonists can be added to prevent metabolism of L-DOPA to dopamine until it reaches the brain, thereby reducing or eliminating emesis caused by systemically administered (e.g., per os) L-DOPA.

Serotonin is found in the enterochromafin cells of the GI tract (approx. 90% of total body serotonin), platelets, and in the CNS. As illustrated in FIG. 6, serotonin and melatonin synthesis are dependent upon BH4, and their synthesis is therefore inhibited in DHEA-treated mammals. However, an alternative biosynthetic mechanism can be employed in which 5-hydroxytryptophan (5HT; CAS 4350-09-8), Vitamin C (Ascorbic acid; CAS 50-81-7), Pyridoxine (pyridoxal phosphate, Vitamin B6; CAS 54-47-7), vitamin B5 (pantothenic acid; CAS 599-54-2) and S-Adenosylmethionine (SAMe; CAS 29908-03-0 can be administered to produce both serotonin and melatonin. The present invention encompasses each of these methods to restore normal levels of dopamine, epinephrine, norepinephrine, melatonin and serotonin in DHEA-treated neutered or aging dogs.

The monoamine part of the reconstitution protocol is thus a mixture of L-DOPA, 5HT, pyridoxine, SAMe, ascorbate, pantothenic acid, and zinc, with or without the addition of a dopamine antagonist. See FIGS. 6 and 8.

For clarification, we will now enumerate our major findings underlying this invention.

Our studies have shown that the levels of the major steroid hormones approach virtually undetectable levels in neutered dogs (FIG. 1).

Our studies show that administration of DHEA to neutered animals stimulates EGSS, and that such administration in neutered animals (with high cancer risk) can restore steroid hormone blood levels to those of intact animals (that have much lower cancer risk). (FIG. 2).

Our studies show that BH4, IPA, NO, one carbon pool products (e.g., AMP, ADP, ATP), ubiquinone, and important monoamines are depleted, and phenylalanine levels are increased, in tissues of neutered dogs undergoing EGSS with DHEA. (FIGS. 3, 4, 5, 6, 7, 8, 9; Table 1).

Our studies show that DHEA-induced EGSS can lead to an auto-inflammatory-like disorder which manifests itself in (1) a rash affecting skin, oral, ocular and other serosal surfaces; (2) fever; (3) monoamine depletion-related sequellae such as motor incoordination and depression; (4) lack of resistance to infections, especially in the ear and eye; (5) increased levels of phenylalanine in serum and tissues; (6) loss of pigmentation (melanin depletion); (7) neuropathy, particularly in the lower limbs, and (8) vasodilation problems attributable to the inability to synthesize NO. These results are tabulated in Table 1.

Our studies show that reconstitution of BH4 and IPA (or mevalonate), addition of an NO donor such as potassium nitrate, addition of folinic acid to maintain one carbon pool metabolism (or the depleted metabolites themselves, e.g. purines, pyrimidines, SAMe), addition of either ubiquinone and/or one or more tocotrienols, and addition of the monoamine reconstitution mix described above, abolish the negative sequellae of DHEA mediated EGSS, permitting normal steroid hormone levels to be restored in the absence of negative side effects.

### BRIEF DESCRIPTION OF FIGURES AND TABLES

FIG. 1. Comparison of testosterone and estrogen levels in male and female dogs, respectively. Analyses were made in serum isolated from 4 intact male and 4 neutered male dogs of mixed breed, and in 3 intact female and 4 neutered female dogs of mixed breed. All were between 12 months and 24 months of age, and between 10 and 25 kg. In male dogs, serum was collected and the method of Schoneshofer and Weber (J Clin Chem Clin Biochem 21(4):231-6, 1983) was utilized for testosterone quantitation, with slight modifications. For female dogs, estrogen was measured in anestrous for intact dogs. For both intact and neutered female dogs, the feces were collected over a period of 30 days, and thereafter the estrogens were extracted and quantitated using the procedure of Hofmann and Mostl (J Reprod Fertil Suppl 57:67-70, 2001). Results are reported as mean±SEM.

FIG. 2. Comparison of testosterone and estrogen in neutered male and female dogs, respectively, before and after DHEA-mediated EGSS. The same neutered dogs from FIG. 1 were utilized. The same methodology was utilized as in FIG. 1. Dogs were exposed to escalating doses of DHEA until normal or near normal levels of testosterone or estradiol were achieved. A dose-dependent relationship was observed. The results in FIG. 2 show that DHEA (10 mg/kg p.o.) increases testosterone and estrogen via EGSS, and that normal or near normal testosterone and estrogen levels can be reconstituted by DHEA-mediated EGSS. Results are reported as the mean±SEM.

FIG. 3. Depletion and reconstitution of BH4, IPA and ubiquinone in dogs treated with DHEA to induce EGSS. BH4 was assayed by the method of Niederwieser, A. et. al. (J Chromatogr. 1984 May 4; 290:237-46). For IPA quantitation, preliminary in vitro studies were performed in which DHEA-treated and control HT-29SF cells were exposed to [³H]-mevalonic acid (saponified; data not shown). Total RNA was then isolated using the guanidinium thiocyanate method, hydrolyzed to free bases and isolated on an Aminex A9 column, with UV detection, as previously described (Nyce, J et. al., Proc Natl Acad Sci USA. 1993 Apr. 1; 90(7):2960-4). Increased incorporation of [³H]-mevalonic acid into isopentenyladenosine peaks was interpreted as reduced pools of free unlabeled IPA into which the [³H]-IPA equilibrated with high specific activity. (See Shultz and Nyce, Cancer Research 51(24):6563-7, 1992). This was borne out in in vivo experiments in neutered dogs treated with DHEA in which buffy coat lymphocytes were extracted and IPA quantitated with UV detection using the same HPLC procedure. In those in vivo studies, total pools of isopentenylated tRNA were indeed found to be depleted. Ubiquinone concentrations were quantitated from buffy coat samples by HPLC via the method of Abe, K. et. al., (J Nutr Sci Vitaminol (Tokyo). 1978; 24(6):555-67). For ease of presentation, pretreatment values for all 3 determinations (BH4, IPA and ubiquinone) were given a value of 1, with treatment values presented as their respective fraction thereof. Neutered male and female dogs had blood or other specimens drawn pretreatment, and then following 90 days of 10 mg/kg DHEA±reconstitution protocol. In this particular experiment, the reconstitution protocol consisted of 2 mg/kg BH4; 2 mg/kg IPA; 1 mg/kg folininc acid; 0.5 mg/kg ubiquinone, ±10 mg/kg mixed tocotrienols; 0.05 mg/kg L-DOPA; 0.1 mg/kg 5HT; 0.2 mg/kg pyridoxal phosphate; 2.5 mg/kg ascorbic acid; 0.25 mg/kg SAMe; 0.5 mg/kg pantothenic acid; and trace zinc. Other concentrations of these metabolites were also found to work. MNIPA, Neutered male dogs, IPA concentrations; FNIPA, female neutered dogs, IPA concentrations; MNBH4, male neutered dogs, BH4 concentrations; FNBH4, female neutered dogs, BH4 concentrations; MNUb, neutered male dogs, Ubiquinone concentrations; FNUb, female neutered dogs, Ubiquinone concentrations; MNUbT, neutered male dogs, Ubiquinone concentrations after reconstitution with Ubiquinone plus tocotrienols; FNUbT, neutered female dogs, ubiquinone concentrations after reconstitution with ubiquinone plus tocotrienols.

FIG. 4. Folate pathway, showing its heavy dependence upon NADPH, and the ability to bypass this NADPH dependence using folinic acid supplementation.

FIG. 5. Depletion of one carbon pool products during DHEA-mediated EGSS, and their reconstitution by folinic acid supplementation. Fifteen ml blood samples were drawn and the buffy coat was isolated by low speed centrifugation in the standard manner. Nucleotides were extracted from 10⁶ lymphocytes and quantitated via HPLC by the method of de Abreu, R A et. al. (J Chromatogr 227(1):45-52, 1982). AMP, ADP and ATP were quantitated. In neutered dogs prior to treatment with DHEA, [AMP]/10⁶ lymphocytes was approx. 50 nM; ADP approx. 300 nM; ATP approx. 350 nM. Then these same dogs were put on a DHEA regimen of 10 mg/kg daily per os for 90 days, ±reconstitution regimen, as above. On day 91, 10-15 ml blood samples were again drawn and nucleotides extracted as before. For ease of presentation, results are expressed as % of pretreatment values. Dark bars, DHEA only. Lighter bars, DHEA+reconstitution regimen. MNATP, neutered male dogs, ATP quantitation; FNATP, neutered female dogs, ATP concentrations; and equivalent for ADP and AMP quantitation.

FIG. 6. Serotonin and melatonin biosynthetic pathway.

FIG. 7. Depletion of serotonin in DHEA-treated dogs (10 mg/kg daily for 90 days), and its reconstitution by supplementation with BH4 and/or the aforementioned monoamine precursor/cofactor mix. The procedure of D'Souza, L and Glueck, H (Thromb Haemost. 1977 Dec. 15; 38(4):990-1001) was used to quantitate serotonin in intestinal biopsies obtained from neutered male and female dogs. Ten mg of intestinal mucosa was analyzed for each animal. The average serotonin extracted and measured from 10 mg of intestinal mucosa was 22.5 umols for neutered male dogs pretreatment, and 20.25 umols for neutered female dogs pretreatment. Results are presented as percentage of values in pretreatment dogs, mean±SEM. MNSer, serotonin levels as quantitated in neutered male dogs; FNSer, serotonin levels as quantitated in female neutered dogs.

FIG. 8. Catecholamine biosynthetic pathway.

FIG. 9. Depletion of norepinephrine in urine of neutered dogs undergoing DHEA-mediated EGSS, and their return to normal values with the addition of the monoamine reconstitution mix. Urinary free norepinephrine was quantitated by the method of Mell, L D and Gustafson, A B (Clin Chem 23(3): 473-6, 1977), using a Waters HPLC system with a C18 reverse phase column and UV detector. 24 hour urine was collected from neutered male and female animals in amber glass containment vessels. To 100 ml of urine was added 1 ml of concentrated HCL as preservative. Pretreatment values for urinary serotonin excretion in neutered males averaged ug/24 hr, and for neutered females, ug/24 hr. These same neutered dogs were then administered DHEA (10 mg/kg)±the reconstitution regimen which included the monoamine mix described above. This treatment continued for 90 days, at which time further urine specimens were collected and assayed for norepinephrine. The data are reported as the mean±SEM. MNNor, norepinephrine levels as quantitated in neutered male dogs; FNNor, norepinephrine levels as quantitated in neutered female dogs.

FIG. 10. Increase in phenylalanine levels in DHEA-treated neutered dogs, and their reduction by supplementation with BH4 in the reconstitution protocol. Before treatment, 10-15 ml blood samples were taken from male and female neutered dogs, and plasma was collected by low speed centrifugation by the standard method. The method of Neckers, L M et. al. (Clin Chem 27(1):146-8, 1981) was used to quantitate plasma phenylalanine concentrations, with fluorometric detection (280 nm excitation, 330 nm emission), and a 10 ul injection volume. MNPhe, phenylalanine concentrations in neutered male dogs. FNPhe, phenylalanine in neutered female dogs.

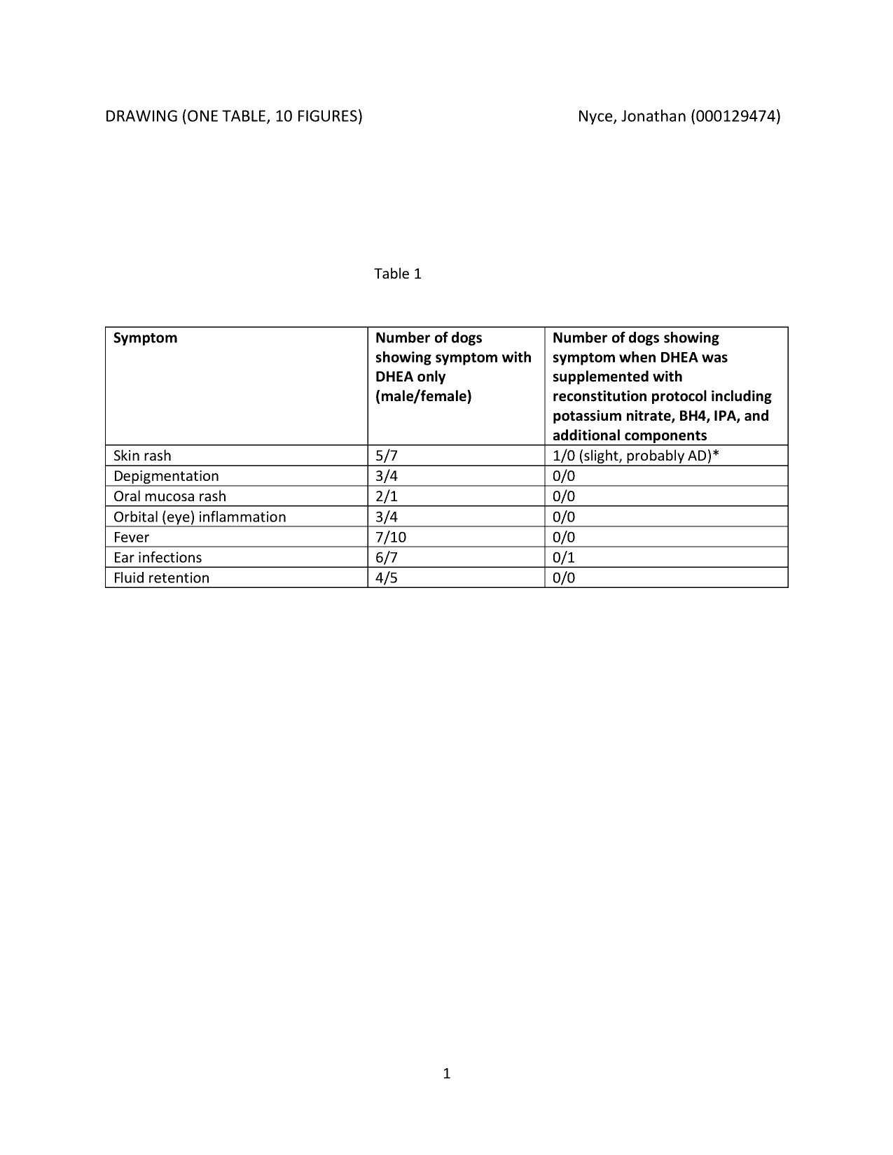

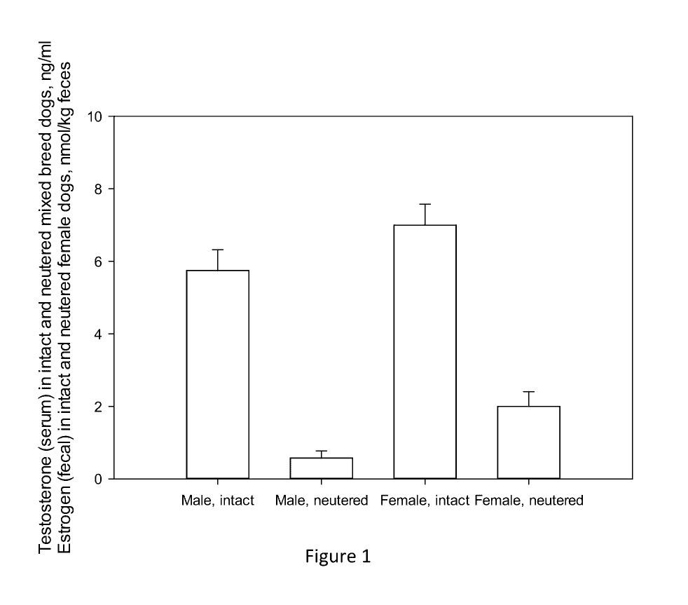

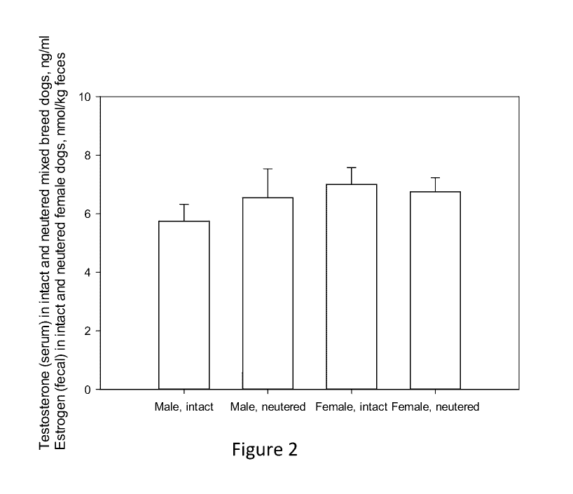

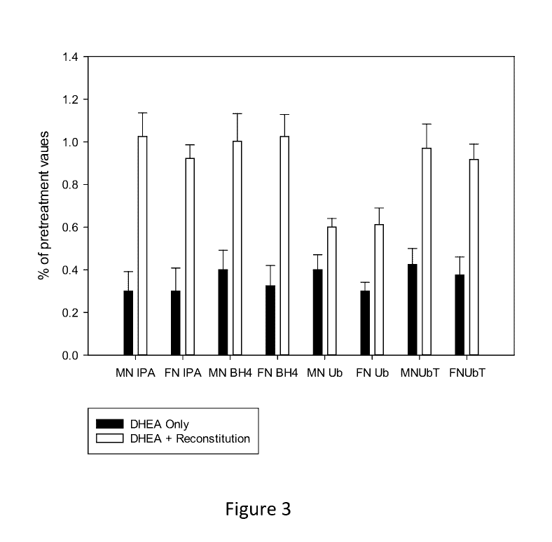

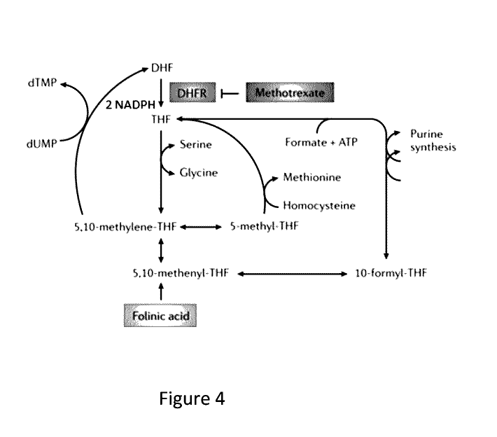

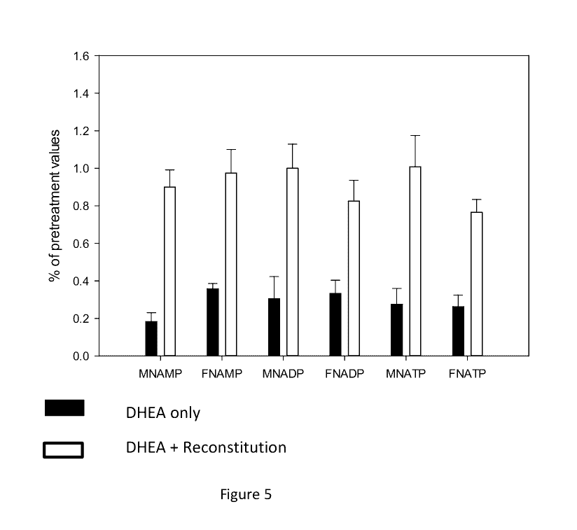

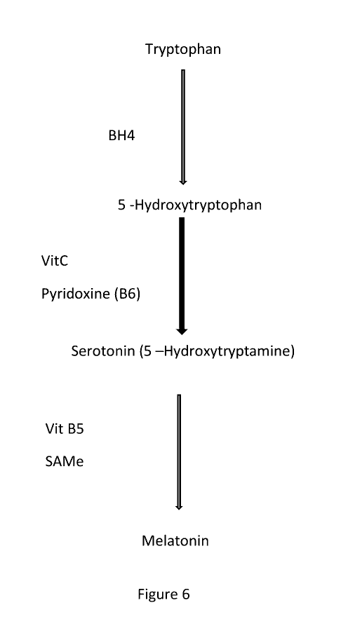

Table 1. Physical symptoms associated with DHEA-mediated EGSS-induced Nitric Oxide (NO) depletion. 10 Male and 10 female neutered mixed breed dogs were administered either DHEA alone (10 mg/kg), or DHEA (10 mg/kg) plus the reconstitution protocol including BH4, 2.5 mg/kg and IPA (2.5 mg/kg) daily for 90 days. A slight predominance of symptoms occurring in female neutered dogs was noted. AD, atopic dermatitis. *This male dog showed slight signs of atypia prior to the initiation of the protocol. This atypia, probably atopic dermatitis, did not clear up with the reconstitution protocol, but neither did it get worse. No other dogs in the study showed any signs of atopic dermatitis or other skin issues prior to initiation of the protocol.

### DETAILED DESCRIPTION OF THE INVENTION

A preferred embodiment of the invention is a pharmaceutical composition of DHEA in sufficient amount to induce EGSS, thereby reconstituting normal or near normal steroid hormone levels in neutered animals, and sufficient BH4, IPA, Potassium Nitrate (or similar nitric oxide donor), folinic acid (or purines, pyrimidines, SAMe), monoamine precursors and cofactors, and ubiquinone and/or tocotrienol to maintain or reconstitute normal or near normal levels of, respectively, BH4, IPA, nitric oxide, one carbon pool metabolites, monoamine precursors and cofactors, and ubiquinone, thereby preventing the negative side effects of EGSS. To effect EGSS and restore steroid hormones to normal or near normal levels, DHEA or its congeners can be administered at a dose of, preferentially, from about 0.1 to 50 mg/kg; more preferentially 0.5 to 30 mg/kg; and most preferentially 1 to 10 mg/kg. DHEA, DHEAS, DHEA sulfatide and any of the salts and derivatives noted above may be manufactured and purified by any of several published methods. For example, the sulfatide can be prepared in high yield (68%) by the reaction of the silver salt of 5-androstene-3β-ol-17-one 3-sulfate with dipalmitoyl α-iodopropylene glycol (Abou-Gharbia, M et. al., J Pharmaceutical Sciences 70:10, 1154-1157, 1981). 7-Keto DHEA and its isomers can be prepared by any of several published procedures (See, for example, USPTO Application US 20070032462 A1). DHEA itself can be manufactured in high yield under mild reaction conditions using the procedure outlined in CN 102212099 A, and by many other methods. DHEA salicylate can be manufactured by the method described in U.S. Pat. No. 5,736,537 A. DHEA and its isomers, derivatives, precursors, metabolites, etc. useful in this invention can be formulated as dry powders; as micronized or otherwise manipulated dry powders to enhance their formulation, encapsulation, compression into tablets, uptake or delivery; as liquids, with or without flavor enhancers and stabilizers; as semi-liquid mixtures, as for example in gel caps. DHEA or its isomers, derivatives, metabolites and precursors can be administered orally as a liquid, a lozenge, a capsule or a tablet. Flavor additives, stabilizers, solubilizing agents and flow enhancers and the like can be used to modify its flavor, activity, solubility and compressibility. DHEA or its isomers, derivatives, metabolites and precursors may also be administered non parenterally by any number of methods including transdermally; as a suppository; by inhalation of respirable formulations either to the lung or nasal cavities; by way of eye drops; sublingually; and by injection by any of the following routes: subcutaneous, intravenous, intraperitoneal, intracranial or intrathecal. Such injections can also be formulated as depot deliveries to increase duration or effect, or to achieve another effect. DHEA or its isomers, derivatives, precursors or metabolites can be formulated as described above either alone or in combination with any or more of the other components of this invention, which include BH4, IPA, one or more nitric oxide donors, folinic acid, monoamine precursors and cofactors, ubiquinone and/or one or more tocotrienols.

To prevent or adequately reduce the effects of BH4 depletion during DHEA-mediated EGSS, BH4 can be administered, as part of the reconstitution mixture, at a dose of between, preferentially, 0.1 and 50 mg/kg; more preferentially between 1 and 30 mg/kg; and most preferentially between 5 and 25 mg/kg. BH4 can be manufactured using any one of several methods. See, for example, U.S. Pat. No. 3,505,329 A; U.S. Pat. No. 8,178,670 B2; U.S. Pat. No. 4,595,752 A; CN101959891 A; WO2012048451 A1; CA2678165 C (Crystalline forms of the dihydrochloride); CN102443006 A (Hydrochloride); U.S. Pat. No. 4,649,197 A (sulfate); U.S. Pat. No. 4,550,109 A (lipoidal derivatives); WO2013152608 A1 (sapropterin dihydrochloride). Preparation of a sulfate of BH4 is described in CA 1250837 A1. BH4 and its isomers, derivatives, and prodrugs useful in this invention can be formulated as dry powders; as micronized or otherwise manipulated dry powders to enhance their formulation, encapsulation, compression into tablets, uptake or delivery; as liquids, with or without flavor enhancers and stabilizers; as semi-liquid mixtures, as for example in gel caps; as respirable particles; and the like. BH4 and its isomers, derivatives, and prodrugs can be administered orally, as a liquid, a lozenge, a capsule or a tablet. Flavor additives, stabilizers, solubilizing agents and flow enhancers and the like can be used to modify its flavor, activity, solubility and compressibility. BH4 and its isomers, derivatives, and prodrugs may also be administered non parenterally by any number of methods including transdermally; as a suppository; by inhalation of respirable formulations either to the lung or nasal cavities; by way of eye drops; sublingually; and by injection by any of the following routes: subcutaneous, intravenous, intraperitoneal, intracranial or intrathecal. BH4 and its isomers, derivatives, and prodrugs can be formulated either alone or in combination with any or all of the other components of this invention, which include DHEA, IPA, one or more nitric oxide donors, folinic acid, monoamine precursors and cofactors, ubiquinone and/or one or more tocotrienols.

To prevent or adequately reduce the effects of IPA depletion during DHEA-mediated EGSS, IPA or its monophosphate (IPAMP) can be administered, as part of the reconstitution mixture, at a dose of between, preferentially, 0.1 and 50 mg/kg; more preferentially between 1 and 25 mg/kg; and most preferentially between 2 and 10 mg/kg. IPA can also be manufactured using any one of several methods including the classical Dimroth rearrangement, and by nucleophilic substitution reactions. (See, for example, Turner, M. L., Thesis, Department of Chemistry, Atlanta University, 1980; Robins, M. L. et. al., Biochemistry 6: 1837-1848, 1967; Rajabi, M. et. al., Nucleic Acid Therapeutics 21 (5), 2011). IPA can also be utilized in the form of its nucleotide (e.g., IPA monophosphate, IPAMP, or di- or tri-phoshate). Natural isoprenoid donors such as farnesol, geraniol, and geranylgeraniol can also replenish the mevalonate pathway to some degree. IPA and its isomers, derivatives, and prodrugs useful in this invention can be formulated as dry powders; as micronized or otherwise manipulated dry powders to enhance their formulation, encapsulation, compression into tablets, uptake or delivery; as liquids, with or without flavor enhancers and stabilizers; as semi-liquid mixtures, as for example in gel caps; as respirable particles; and the like. IPA and its isomers, derivatives, and prodrugs can be administered orally, as a liquid, a lozenge, a capsule or a tablet. Flavor additives, stabilizers, solubilizing agents and flow enhancers and the like can be used to modify its flavor, activity, solubility and compressibility. IPA and its isomers, derivatives, and prodrugs may also be administered non parenterally by any number of methods including transdermally; as a suppository; by inhalation of respirable formulations either to the lung or nasal cavities; by way of eye drops; sublingually; and by injection by any of the following routes: subcutaneous, intravenous, intraperitoneal, intracranial or intrathecal. IPA and its isomers, derivatives, and prodrugs can be formulated either alone or in combination with any or all of the other components of this invention, which include DHEA, BH4, one or more nitric oxide donors, folinic acid (or purines, pyrimidines, SAMe), monoamine precursors and cofactors, ubiquinone and/or one or more tocotrienols.

To prevent or adequately reduce the effects of nitric oxide depletion during DHEA-mediated EGSS, potassium nitrate (or sodium nitrite) can be administered, as part of the reconstitution mixture, at a dose of between, preferentially, 1 and 50 mg/kg; more preferentially between 2 and 25 mg/kg; and most preferentially between 3 and 15 mg/kg. Potassium nitrate is available commercially in human pharmaceutical grade. Other nitric oxide donors are also enveloped by our invention, including but not limited to furoxan-based DHEA hybrid molecules as described in the literature (See Huang, Y et. al., Steroids 2015, May 22, pii:S0039-128X(15)00149-X); sodium nitrite; nitric oxide gas; S-nitrosothiol, diazeniumdiolate; NONOate; furoxan; nitroaspirin; and organic nitrate (see Miller, M R and Megson, I L, Br J Pharmacol 151(3):305-321, June, 2007). Nitric oxide donors, derivatives, and prodrugs useful in this invention can be formulated as dry powders; as micronized or otherwise manipulated dry powders to enhance their formulation, encapsulation, compression into tablets, uptake or delivery; as liquids, with or without flavor enhancers and stabilizers; as semi-liquid mixtures, as for example in gel caps; as respirable particles; and the like. Nitric oxide donors, derivatives, and prodrugs can be administered orally, as a liquid, a lozenge, a capsule or a tablet. Flavor additives, stabilizers, solubilizing agents and flow enhancers and the like can be used to modify its flavor, activity, solubility and compressibility. Nitric oxide donors, derivatives, and prodrugs may also be administered non parenterally by any number of methods including transdermally; as a suppository; by inhalation of respirable formulations either to the lung or nasal cavities; by way of eye drops; sublingually; and by injection by any of the following routes: subcutaneous, intravenous, intraperitoneal, intracranial or intrathecal. Nitric oxide donors, derivatives, and prodrugs can be formulated either alone or in combination with any or all of the other components of this invention, which include DHEA, BH4, IPA, folinic acid, monoamine precursors and cofactors, ubiquinone and/or one or more tocotrienols.

To prevent or adequately reduce the effects of ubiquinone depletion during DHEA-mediated EGSS, tocotrienols, either individual isomers or as mixtures of isomers, can be administered, as part of the reconstitution mixture, at a dose of between, preferentially, 1 and 50 mg/kg; more preferentially between 2 and 30 mg/kg; and most preferentially between 5 and 25 mg/kg. Tocotrienols can be manufactured using any one of several methods including isolation from palm and other oils (Ng, M H et. al., Lipids. 2004 October; 39(10):1031-5; Luidy Rodriguez Posada et. al., Separation and Purification Technology Volume 57, Issue 2, 15 October 2007, Pages 220-229), rice bran (Qureshi A A, et. al., J Agric Food Chem. 2000 August; 48(8):3130-40), and other sources (See N. Othman, et. al., 2010. Journal of Applied Sciences, 10: 1187-1191). The palmitate, stearate and 4-phenylbenzoate esters of D-gamma-tocotrienol can also be synthesized and purified using published procedures (See U.S. Pat. No. 5,670,668). Tocotrienols (α, β, γ, δ, separately or in any combination), their derivatives, and prodrugs useful in this invention can be formulated as dry powders; as micronized or otherwise manipulated dry powders to enhance their formulation, encapsulation, compression into tablets, uptake or delivery; as liquids, with or without flavor enhancers and stabilizers; as semi-liquid mixtures, as for example in gel caps; as respirable particles; and the like. Tocotrienols (α, β, γ, δ, separately or in any combination), their derivatives, and prodrugs can be administered orally, as a liquid, a lozenge, a capsule or a tablet. Flavor additives, stabilizers, solubilizing agents and flow enhancers and the like can be used to modify its flavor, activity, solubility and compressibility. Tocotrienols (α, β, γ, δ, separately or in any combination), their derivatives, and prodrugs may also be administered non parenterally by any number of methods including transdermally; as a suppository; by inhalation of respirable formulations either to the lung or nasal cavities; by way of eye drops; sublingually; and by injection by any of the following routes: subcutaneous, intravenous, intraperitoneal, intracranial or intrathecal. Tocotrienols (α, β, γ, δ, separately or in any combination), their derivatives, and prodrugs can be formulated either alone or in combination with any or all of the other components of this invention, which include DHEA, BH4, IPA, one or more nitric oxide donors, monoamine precursors and cofactors, and ubiquinone.

To prevent or adequately reduce the effects of ubiquinone depletion during DHEA-mediated EGSS, ubiquinone can be administered, as part of the reconstitution mixture, at a dose of between, preferentially, 0.1 and 20 mg/kg; more preferentially between 0.5 and 10 mg/kg; and most preferentially between 1 and 5 mg/kg. Ubiquinone is available in pharmaceutical grade purity or can be manufactured by any one of several methods. For example, optically pure ubiquinone can be synthesized in bulk by the process described in U.S. Pat. No. 6,506,915 B1, and U.S. Pat. No. 6,686,485 and a publication Mahendra, M., et. al. (International Journal of Chemical Sciences and Research ISSN Print: 2249-0329 Website: http://www.ijcsr.co.in/). Both describe a semi synthetic procedure using solanesol derived from tobacco waste as the starting material for the sterospecific synthesis of ubiquinone. Additionally, large scale synthesis of ubiquinone in high yield can be performed by an SN2′-type nucleophilic displacement reaction between copper-catalyzed Grignard reagent and allylic acetate as reported by Wang, Fen et. al. (Letters in Organic Chemistry, Volume 3, Number 8 August 2006, pp. 610-612(3)). Ubiquinone, its derivatives, and prodrugs useful in this invention can be formulated as dry powders; as micronized or otherwise manipulated dry powders to enhance their formulation, encapsulation, compression into tablets, uptake or delivery; as liquids, with or without flavor enhancers and stabilizers; as semi-liquid mixtures, as for example in gel caps; as respirable particles; and the like. Ubiquinone, its derivatives, and prodrugs can be administered orally, as a liquid, a lozenge, a capsule or a tablet. Flavor additives, stabilizers, solubilizing agents and flow enhancers and the like can be used to modify its flavor, activity, solubility and compressibility. Ubiquinone, its derivatives, and prodrugs may also be administered non parenterally by any number of methods including transdermally; as a suppository; by inhalation of respirable formulations either to the lung or nasal cavities; by way of eye drops; sublingually; and by injection by any of the following routes: subcutaneous, intravenous, intraperitoneal, intracranial or intrathecal. Ubiquinone, its derivatives, and prodrugs can be formulated either alone or in combination with any or all of the other components of this invention, which include DHEA, BH4, IPA, one or more nitric oxide donors, monoamine precursors and cofactors, and one or more Tocotrienols.

To prevent or adequately reduce the effects of depletion of folate pathway products during DHEA-mediated EGSS, folinic acid can be administered, as part of the reconstitution mixture, at a dose of between, preferentially, 0.01 and 15 mg/kg; more preferentially between 0.1 and 10 mg/kg; and most preferentially between 0.3 and 5 mg/kg. Folinic acid can be synthesized and purified by many methods in the public domain, including that of Temple, C, Jr et. al. (J Med Chem. 22(6):731-4, 1979; Sato, J K et. al., Anal Biochem. 154(2):516-24, 1986; U.S. Pat. No. 5,134,235 A), and by methods still under patent, for example U.S. Pat. No. 8,633,202 B2. As synthesized folinic acid exists as a mixture of optical isomers, these optical isomers can be separated by the method described in U.S. Pat. No. 5,599,931 A, and by other methodologies. Stabilized aqueous preparations of folinic acid, for example, for injection, can be obtained through the method described in U.S. Pat. No. 6,613,767 B1 and European Patent EP1640008 B1. The sodium salt of folinic acid can be prepared according to the method described in U.S. Pat. No. 6,160,116 A. folinic acid, its derivatives, salts and prodrugs useful in this invention can be formulated as dry powders; as micronized or otherwise manipulated dry powders to enhance their formulation, encapsulation, compression into tablets, uptake or delivery; as liquids, with or without flavor enhancers and stabilizers; as semi-liquid mixtures, as for example in gel caps; as respirable particles; and the like. Folinic acid, its derivatives, and prodrugs can be administered orally, as a liquid, a lozenge, a capsule or a tablet. Flavor additives, stabilizers, solubilizing agents and flow enhancers and the like can be used to modify its flavor, activity, solubility and compressibility. Folinic acid, its derivatives, and prodrugs may also be administered non parenterally by any number of methods including transdermally; as a suppository; by inhalation of respirable formulations either to the lung or nasal cavities; by way of eye drops; sublingually; and by injection by any of the following routes: subcutaneous, intravenous, intraperitoneal, intracranial or intrathecal. Folinic acid, its derivatives, and prodrugs can be formulated either alone or in combination with any or all of the other components of this invention, which include DHEA, BH4, IPA, one or more nitric oxide donors, monoamine precursors and cofactors, ubiquinone and/or one or more Tocotrienols.

To prevent or adequately reduce the effects of monoamine depletion during DHEA-mediated EGSS, L-DOPA can be administered at a dose of, preferentially, from 0.1 mg/kg to 25 mg/kg; more preferentially, 0.5 mg/kg to 15 mg/kg; and most preferentially from 1 mg/kg to 10 mg/kg. 5-HT can be administered at a dose of, preferentially, 0.1 mg/kg to 25 mg/kg; more preferentially, 0.5 mg/kg to 15 mg/kg; and most preferentially, 1 mg/kg to 10 mg/kg. Pyridoxine can be administered at a dose of, preferentially, 0.05 mg/kg to 5 mg/kg; more preferentially, 0.09 mg/kg to 2.5 mg/kg; and most preferentially, 0.5 mg/kg to 1 mg/kg; SAMe can be administered at a dose of, preferentially, 1 mg/kg to 100 mg/kg; more preferentially, 2 mg/kg to 50 mg/kg; and most preferentially, 5 mg/kg to 10 mg/kg. Ascorbic acid can be administered at a dose of, preferentially, 1 mg/kg to 100 mg/kg; more preferentially, 2 mg/kg to 50 mg/kg; and most preferentially, 5 mg/kg to 25 mg/kg. Pantothenic acid can be administered at a dose of, preferentially, 0.1 mg/kg to 100 mg/kg; more preferentially, 0.5 mg/kg to 50 mg/kg; and most preferentially, 1 mg/kg to 10 mg/kg. Zinc may be administered at a dose of, preferentially, 0.05 mg/kg to 10 mg/kg; more preferentially, 0.1 mg/kg to 5 mg/kg; and most preferentially, 0.5 mg/kg to 2.5 mg/kg. Monoamine precursors and cofactors, including L-DOPA, 5HT, pyridoxine, SAMe, ascorbate, and pantothenic acid, and zinc, can each be purchased commercially in highly purified form, or can be synthesized and purified by many methods in the public domain. Highly purified L-DOPA is available commercially, and can be manufactured according to standard chemical methods employing asymmetric synthesis and metal catalysts, or electroenzymatically employing a tyrosinase-immmobilized cathode under the reduction potential of DOPAquinone (See Min, K et al, J. Biotechnol 146(1-2):40-44, 2010). 5-HT is available commercially in highly purified form. It can be synthesized by many published methods (See, for example, Frangatos, G and Chubb, F, Can J Chem 37:1374-76, 1959), including a newly reported cofactor regeneration process using modified L-phenylalanine 4-hydroxylase of Chromobacterium violaceum. (See Hara, R and Kino, K, AMB Express 3:70, 2013). Pyridoxal phosphate is available commercially in highly purified form. SAMe is available commercially in highly purified form, of which the dihydrochloride salt is particularly useful. Ascorbic acid is also available commercially in highly purified form, as are pantothenic acid and various formulations of zinc. Each of these monoamine precursors or cofactors, their derivatives, salts and prodrugs useful in this invention can be formulated as dry powders; as micronized or otherwise manipulated dry powders to enhance their formulation, encapsulation, compression into tablets, uptake or delivery; as liquids, with or without flavor enhancers and stabilizers; as semi-liquid mixtures, as for example in gel caps; as respirable particles; and the like. Each of these monoamine precursors or cofactors, their derivatives, and prodrugs can be administered orally, as a liquid, a lozenge, a capsule or a tablet. Flavor additives, stabilizers, solubilizing agents and flow enhancers and the like can be used to modify its flavor, activity, solubility and compressibility. Each of these monoamine precursors or cofactors, their derivatives, and prodrugs may also be administered non parenterally by any number of methods including transdermally; as a suppository; by inhalation of respirable formulations either to the lung or nasal cavities; by way of eye drops; sublingually; and by injection by any of the following routes: subcutaneous, intravenous, intraperitoneal, intracranial or intrathecal. Each of these monoamine precursors or cofactors, their derivatives, and prodrugs can be formulated either alone or in combination with any or all of the other components of this invention, which include DHEA, BH4, IPA, one or more nitric oxide donors, folinic acid (or adenine (or hypoxanthine) and uracil, their nucleosides or nucleotides), ubiquinone and/or one or more Tocotrienols.

As used in the context of this invention, co-administration refers to temporal proximity. Thus, the agents described as “co-administered” may be administered exactly together; they may be delivered one or more before the other(s), so as to prevent the onset of negative side effects; they may be delivered one or more after the other(s), for example, to cost effectively supply them only when the need becomes more pressing; or some combination of the above.

Examples of binders are gum tragacanth, acacia, starch, gelatin, and biological degradable polymers such as homo- or co-polyesters of dicarboxylic acids, alkyiene glycols, polyalkyiene glycols and/or aliphatic hydroxyl carboxylic acids; homo- or co-polyamides of dicarboxylic acids, alkylene diamines, and/or aliphatic amino carboxylic acids; corresponding poly-ester-polyamide-co-polymers, polyanhydrides, polyorthoestens, polyphosphazene and poly-carbonates. The biological degradable polymers may be linear, branched or cross-linked.

Specific examples are poly-glycolic acid, poly-lactic acid, and poly-d, l-lactide/glycolide. Other examples for polymers are water-soluble polymers such as polyoxalkylenes (polyoxethylene, polyoxapropylene and mixed polymers thereof, poly-acrylamides and hydroxylalkylated polyacrylamides, poly-maleic acid and esters or -amides thereof, poly-acrylic acid and esters or -amides thereof, poly-vinylalcohol and esters or -ethers thereof, poly-vinylimidazole, poly-vinylpyrrolidon, and natural polymers like chitosan.

Examples for excipients are phosphates such as dicalcium phosphate.

Examples for lubricants are natural or synthetic oils, fats, waxes, or fatty acid salts like magnesium stearate, sesame oil, olive oil, coconut oil, tocopherols and the like.

Surfactants may be ionic, anionic, amphoteric or neutral. Examples for surfactants are lecithin, phospholipids, octyl sulfate, decyl sulfate, dodecyl sulfate, tetradecyl sulfate, hexadecyl sulfate and octadecyl sulfate, Na oleate or Na caprate, 1-acyiaminoethane-2-sulfonic acids, such as 1-octanoylaminoethane-2-sulfonic acid, 1-decanoylaminoethane-2-sulfonic acid, 1-dodecanoylaminoethane-2-sulfonic acid, 1-tetradecanoylaminoethane-2-sulfonic acid, 1-hexadecanoylaminoethane-2-sulfonic acid, and 1-octadecanoylamino-ethane-2-sulfonic acid, and taurocholic acid and taurodeoxycholic acid, bile acids and their salts, such as cholic acid, deoxycholic acid and sodium glycocholates, sodium caprate or sodium laurate, sodium oleate, sodium lauryl sulphate, sodium cetyl sulphate, sulfated castor oil and sodium dioctylsulfosuccinate, cocamidopropylbetaine and laurylbetaine, fatty alcohols, cholesterols, glycerol mono- or -distearate, glycerol mono- or -dioleate and glycerol mono-or -dipalmitate, and polyoxyethylene stearate.

Examples for sweetening agents are sucrose, fructose, lactose, sodium saccharine, Steviol glycosides, or aspartame.

Examples for flavoring agents are bacon, beef, chicken, peppermint, oil of wintergreen or fruit flavors like cherry or orange flavor.

Examples for coating materials are gelatin, wax, shellac, sugar or biological degradable polymers.

Examples for preservatives are methyl or propylparabens, sorbic acid, chlorobutanol, sodium nitrite, potassium nitrate, phenol, butylated hydroxytoluene, butylated hydroxyanisole and thimerosal.

Examples for thickeners are synthetic polymers, fatty acids and fatty acid salts and esters and fatty alcohols.

Examples for antioxidants are vitamins, such as vitamin A, vitamin C, vitamin D or vitamin E, tocopherols, tocotrienols, quercetin, curcumin, L-cysteine, L-acetyl cysteine, sesamin, sesamol, vegetable extracts or fish oils.

Examples for liquid carriers are water, alcohols such as ethanol, glycerol, propylene glycol, liquid polyethylene glycols, triacetin and oils. Examples for solid carriers are talc, clay, micro-crystalline cellulose, silica, alumina and the like.

The formulation according to the invention may also contain isotonic agents, such as sugars, buffers or sodium chloride.

The hydrate form according to the invention may also be formulated as effervescent tablet or powder, which disintegrate in aqueous environment to provide a drinking solution.

Slow release formulations may also be prepared from the polymorph according to the invention in order to achieve a controlled release of the active agent. The formulation may be embedded for this purpose in a polymer matrix of a biological degradable polymer, a water-soluble polymer or a mixture of both, and optionally suitable surfactants. Embedding can mean in this context the incorporation of microparticles in a matrix of polymers. Controlled release formulations are also obtained through encapsulation of dispersed micro-particles or emulsified micro-droplets via known dispersion or emulsion coating technologies.

### United States Patents Cited

U.S. Pat. No. 5,567,696 A, 1996, McGuire, J L, Van Vollenhoven, R F, and Engelman, E G, Treatment of systemic lupus erythematosus with dehydroepiandrosterone
U.S. Pat. No. 7,893,044 B2, 2011, Nyce, Jonathan W. Composition and method for altering levels of or sensitivity to adenosine with analogs of dehydroepiandrosterone
U.S. Pat. No. 5,527,789 A, 1996, Nyce, Jonathan W. Method of inhibiting carcinogenesis by treatment with dehydroepiandrosterone and analogs thereof
U.S. Pat. No. 6,087,351, 2000, A Nyce, Jonathan W, Method for reducing adenosine levels with a dehydroepiandrosterone and optionally a ubiquinone
U.S. Pat. No. 6,670,349 B1, 2003, Nyce, Jonathan, Composition & method for altering levels of or sensitivity to adenosine with a dehydroepiandrosterone &/or a ubiquinone
U.S. Pat. No. 7,456,161 B2, 2008, Jonathan W Nyce, Use of DHEA and DHEA-sulfate for the treatment of chronic obstructive pulmonary disease
U.S. Pat. No. 5,134,235 A, 1992, Mueller, H R et al, Process for separating folinic acid
U.S. Pat. No. 8,633,202 B2, Jequier, P, 2014, Crystalline levofolinic acid and process for its preparation
U.S. Pat. No. 5,599,931 A, Ripa, G et. al., 1997, Process for separating stereoisomers of folinic acid
U.S. Pat. No. 6,613,767 B1, Nijkerk, A J and Vermeer, J M P, 2003, Stable aqueous folinate solution
U.S. Pat. No. 6,160,116 A, Mueller, H R et al., 2000, Sodium salt of (6S)-folinic acid

### Foreign Patents Cited

WO 1996025164 A1, 1995 Use of dehydroepiandrosterone to treat depression. Goodyer, Ian Michael, and Joseph, Herbert (ABSTRACT: Dehydroepiandrosterone (DHEA) is indicated in the treatment of depression. Clinical data show a statistically significant association between blood levels of DHEA, abnormally low (which can be determined by measuring the salivary DHEA), especially in the morning, and major depression, diagnosed based on recognized clinical criteria. The administration of DHEA increased serum levels of DHEA.)
WO 1998032445 A1, 1998, Brian Berger, Samuel S C Yen, Use of dehydroepiandrosterone to treat primary adrenal insufficiency and Addison's disease (ABSTRACT: The present invention provides a method of treating an individual with primary adrenal insufficiency, comprising the step of administering an effective amount of dehydroepiandrosterone to said individual. Also provided is a method of treating an individual with adrenal failure secondary to dysfunctions of the hypothalamus and/or pituitary gland, comprising the step of administering an effective amount of dehydroepiandrosterone to said individual and a method of treating an individual with adrenal insufficiency due to acquired human immunodeficiency syndrome (AIDS), comprising the step of administering an effective amount of dehydroepiandrosterone to said individual).
WO 2002043737 A1, Zenk, J L and Zenk, R, 2002, Treatment of chronic fatigue syndrome and fibromyalgia syndrome (ABSTRACT: Chronic fatigue syndrome (CFS) and fibromyalgia syndrome (FMS) can be treated by the administration of Δ5-androstene-3β-ol-7,17-dione and metabolizable precursors thereof).
WO 2003002123 A3, Van Der Hoop, Roland Gerritsen, 2003, Therapeutic combinations for the treatment of hormone deficiencies (ABSTRACT: The present invention relates to methods of treating, preventing, or reducing the risk of developing a male or female menopause disorder or symptom in a mammal by administering to the mammal a sex hormone binding globulin synthesis inhibiting agent and one or more steroids, including, for example, an androgen or an estrogen; to combinations for treating, preventing or reducing the risk of developing a male or female menopause disorder or symptom in a mammal; and to compositions for treating, preventing, or reducing the risk of developing a male or female menopause disorder or symptom in a mammal, where the composition comprises a sex binding globulin synthesis inhibiting agent and one or more steroids, including, for example, an androgen or an estrogen. In addition, the methods, combinations and compositions may be used in conjunction with other pharmaceutical agents aimed at improving sexual performance or impotence, increasing libido, or treating erectile dysfunction, such as VIAGRA®, to enhance their effectiveness).
EP 1640008 B1, Niyaji, T N P et. al., 2011, A stabilized aqueous (6S)-folinic acid preparation for injection. (Abstract: The present invention relates to a stabilized aqueous preparation for injection containing 5-formyl-(6S)-tetrahydrofolic add or a pharmacologically acceptable salt thereof as an active ingredient).

### OTHER CITATIONS

Abe, K. et. al., (J Nutr Sci Vitaminol (Tokyo). 1978; 24(6):555-67
Ahlem C N, et. al., Steroids. 2011 June; 76(7):669-74
Aizawa K et al, Am J Physiol Endocrinol Metab. 2007 February; 292(2):E577-84. Epub 2006 Oct. 3
Basualto-Alarcón C, et al, Front Endocrinol (Lausanne). 2014 Dec. 18; 5:217
Baliga, R S, et al, Respiratory and Critical Care Medicine, Abstract Issue, B63, Experimental models in pulmonary hypertension, American Thoracic Society International Conference Abstracts, 2012
Belanger B. et. al., Journal of Steroid Biochemistry 32 695-698, 1989
Bentinger, M et. al. (Biofactors, 2008; 32 (1-4):99-111
Bifulco, M. Malfitano, A M, Proto, M C et al. Anticancer Agents Med Chem 2008, 8(2): 2000-2004
Borsi, J D et al, Pediatr Hematol Oncol. 1990;7(4):347-63; folinic acid CAS 1492-18-8
Callewaert F, Boonen S, Vanderschueren D. Trends Endocrinol Metab. 2010 February; 21(2):89-95
Caso, Francesco et al., Int J. Rheumatol 2013, Oct. 24
Chamouni A, Oury F, Arch Biochem Biophys. 2014 Nov. 1; 561:147-53
Coleman, J W, Int Immunopharmacol. 2001 August; 1(8):1397-406
Cunningham G R, Asian J Androl. 2015 March-April; 17(2):192-6
Cutler G B Jr, et al., Endocrinology. 1978 December; 103(6):2112-8
de Abreu, R A et. al., J Chromatogr 227(1):45-52, 1982
Dillon, J S, Curr Drug Targets Inflamm Allergy. 2005 June; 4(3):377-85
D'Souza, L and Glueck, H (Thromb Haemost. 1977 Dec. 15; 38(4):990-1001
Hayashi F., Carcinogenesis. 1994 October; 15(10):2215-9
Hofmann and Mostl (J Reprod Fertil Suppl 57:67-70, 2001
Hudson, T. S. et al, Carcinogenesis 33(6):1225-1230, 2012
Huneeus, V Q, Wiley, M H and Siperstein, M D, PNAS USA October 1980: 77(10):5842-5846
Huneeus, V Q, Wiley, M H and Siperstein, M D, PNAS USA October 1980: 77(10):5842-5846
Inoue, Takayoshi et al, Mol Cell Endocrinol 2012 Oct. 22; 362(1-2):19-28. Epub 2012 May 22
Kapatos, G., The neurobiology of tetrahydrobiopterin biosynthesis, IUBMB Life 65(4):323-33, 2103
Khorram, O et al, J Gerontol A Biol Sci Med Sci. 1997 January; 52(1):M1-7
Khorrami A, et. al., Drug Res (Stuttg). 2014 April; 64(4):177-81. doi: 10.1055/5-0033-1354374. Epub 2013 Sep. 11
Koike, H. et al Neurology. 84(10):1026-33, 2015; doi: 10.1212/WNL.0000000000001343. Epub 2015 Feb. 6
Kryukov, G. V. et al, J. Biol. Chem. 274, 33888-33897, 1999
Labrie F, Dupont A & Belanger A. In Important Advances in Oncology, pp 193-217, 1985. Eds V T de Vita, S Hellman & S A; Rosenberg. Philadelphia: J. B. Lippincott
Labrie F, Belanger A, Cusan L & Candas B, Journal of Clinical Endocrinology and Metabolism 82:2403-2409
Labrie, F et al, Front Neuroendocrinol. 2001 July; 22(3):185-212
Laten, H M and Zahareas-Doktor, S PNAS USA February 1985; 82(4):1113-1115
Leone R D, Lo Y C, and Powell J D, Comput Struct Biotechnol J. 2015 Apr. 8; 13:265-72. doi: 10.1016/j.csbj.2015.03.008. eCollection 2015
Liu Q et al, Am J Pathol. 2012 August; 181(2):452-62
Lotti, V J and Clark, C, Eur J Pharmacol. 1974 March; 25(3):322-5
Mattmiller, S A et al, J. Nut Biochem 25(6):647-54, 2014
Mell, L D and Gustafson, A B (Clin Chem 23(3): 473-6, 1977
Mongillo P et al, Res Vet Sci. 2014 February; 96(1):33-8
Morgentaler A, Asian J Androl. 2015 January-February; 17(1):26-31
Nagasakin, Y. et al., Pediatr Res. 1999 April; 45(4 Pt 1):465-73
Nakamaru-Ogiso E et. al., J Bioenerg Biomembr. 2014 August;46(4):269-77. doi: 10.1007/510863-014-9557-9. Epub 2014 Jul. 31
Neckers, L M et. al. (Clin Chem 27(1):146-8, 1981
Ney D M, Blank R D, and Hansen K E, Curr Opin Clin Nutr Metab Care. 2014 January; 17(1):61-8
Niederwieser, A. et. al. (J Chromatogr. 1984 May 4; 290:237-46
Nyce, J et.al., Proc Natl Acad Sci USA. 1993 Apr. 1; 90(7):2960-4
Nyce J W et al, Carcinogenesis. 1984 January; 5(1):57-62
Odell W D, Parker L N, Endocr Res. 1984-1985; 10(3-4):617-30
Olekhnovitch, R et al, J. Clin Invest 124(4):1711-1722, 2014
Oury F, Ann N Y Acad Sci. 2012 July; 1260:1-7
Pan, L et al, Brit Med J Case Rep bcr0320113927, 2011
Perrier, J F and Cotel, F, Curr Opin Neurobiol. 2014 Dec. 29; 33C:1-7. doi: 10.1016/j.conb.2014.12.008
Rohrmann, Sabine et al, Clin Endocrinol (Oxf) 2011 August, 75(2):232-239
Rissetto, K et al, J Am Anim Hosp Assoc. 2011 January-February; 47(1):28-36
Rohrmann, S., et. al., Clin Endocrinol (Oxf). 2011 August; 75(2): 232-239. doi: 10.1111/j.1365-2265.2011.04043.x
Salerni, S. et al, Eur J Clin Invest. 2015 Apr. 3
Sathish V, Martin Y N, Prakash Y S, Pharmacol Ther. 2015 Jan. 14
Seeher, S. et al, Biochem J. 462(1):67-75, 2014
Stot, Crister A, Karel Dolezal, Anders Nordstrom et al. PNAS USA Dec. 19, 2000; 97(26):14778-14783
Scriver, C R and Clow, C L, Annu Rev Genet. 1980; 14:179-202
Sanford, Mark; Keating, Gillian M. (2009). “Sapropterin”. Drugs 69 (4): 461-76 doi:10.2165/00003495-200969040-00006. PMID 19323589
Sharma, R and Davidoff, M N, Oxidative stress and endothelial dysfunction in heart failure, Congest Heart Fail. 2002 May-June; 8(3):165-72
Schalinskel, K L and Steele, R D Carcinogenesis vol. 17 no. 8 pp. 1695-1700, 1996
Shuto, K et. al. (J Pharmacobiodyn. 1980 December; 3(12):709-14
Schoneshofer and Weber (J Clin Chem Clin Biochem 21(4):231-6, 1983
Shultz and Nyce, Cancer Research 51(24):6563-7, 1992
Sorenmo K U, Goldschmidt M, Shofer F, Ferrocone J. Vet Comparative Oncology.2003 March; 1 (1): 48
Sorwell, K G and Urbanski, H F, J. Neuroendocrin 2013, 25(11):1062-1069, November 2013
Spinola M et al, Oncogene. 2005 Aug. 18; 24(35):5502-9
Srinath, R et al, J Clin Endocrinol Metab. 2015 April; 100(4):1602-8
Strisciuglio P and Concolino D, Metabolites. 2014 Nov. 4; 4(4):1007-17. doi: 10.3390/metabo4041007
Teske E, Naan E C, van Dijk E M, vanGarderen E, Schalken J A. Mol Cell Endocrinol. 2002 Nov. 29; 197(1-2):251-255
Torres de la Riva, G et al, PLoS One, 2013, 8(2):e55937
Tsuji, P. A. et al, PLoS One, 2015, 10(4):e0124487
van der Burgh, R et al, Clin Immunol 147(3):197-206, 2013
van der Molen, H J, et al. Biochim Biophys Acta, 1971 Nov. 5; 248(2):343-62
van Spronsen F J¹, Enns G M, Mol Genet Metab. 2010;99 Suppl 1:S90-5. doi: 10.1016/j.ymgme.2009.10.008
Villamil, J A, et. al., J Cancer Epidemiol 2009:591753
Widstrom, Richard L. Seminars in Reproductive Medicine 12/2004; 22(4):289-98; Labrie, F et al, Journal of Endocrinology (2005) 187, 169-196
Williams, John, Lipids, 04/2000; 35(3):325-31

1. A method for restoring steroid hormone levels in neutered animals comprising administration of (a) Dehydroepiandrosterone (DHEA, Formula 1) or a congener thereof (e.g., formulae 1 a-f) in an amount sufficient to induce Extra Gonadal Steroid Synthesis (EGSS), and (b) a reconstitution mixture to prevent side effects of DHEA-induced NADP(H) depletion during EGSS.

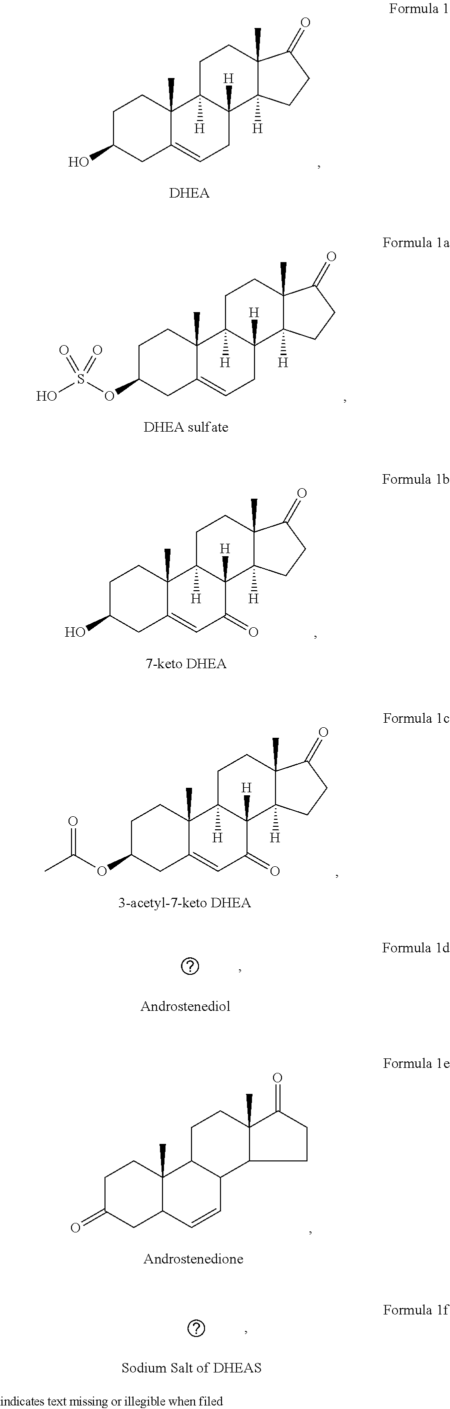

2. The substance(s) of claim 1 administered as an oral, injectable (intravenous, intratumor, subcutaneous, intraperitoneal, intracranial or intrathecal), inhalable, aerosolized, respirable, sublingual, topical (including ophthalmic, otic), transdermal, or suppository, in amounts sufficient to replenish steroid hormone levels in neutered mammals.

3. The method of claim 1, comprising Tetrahydrobiopterin (BH4, Formula 2) or sepiapterin (Formula 2b) as a component of the reconstitution mixture co-administered with the substance(s) of claim 1, with BH4 administered in amounts sufficient to prevent side effects associated with its depletion during DHEA-mediated EGSS.

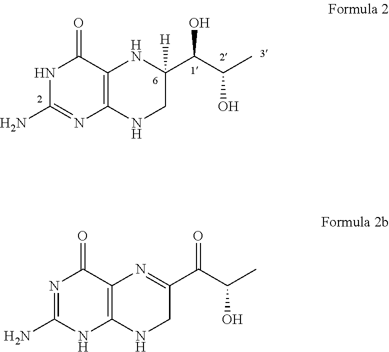 [SMILES: C[C@H](O)[C@H](O)[C@H]1CNc2nc(N)[nH]c(=O)c2N1 ; C[C@H](O)C(=O)C1=Nc2c([nH]c(N)nc2=O)NC1]

4. The substance(s) of claim 3 administered as an oral, sublingual, injectable (intravenous, intratumor, subcutaneous, intraperitoneal, intracranial or intrathecal), inhalable, aerosolized, respirable, topical (including ophthalmic, otic), transdermal, or suppository in amounts sufficient to prevent side effects of BH4 depletion during EGSS performed according to the method of claim 1, in a mammal in need of such treatment.

5. The method of claim 1, comprising administration of N6-Isopentenyladenosine (IPA, Formula 5), IPA monophosphate, or congener (e.g., formula 3b) as a component of the reconstitution mixture co-administered with the substance(s) of claim 1, IPA administered in amounts sufficient to maintain normal levels of isopentenylated tRNA.

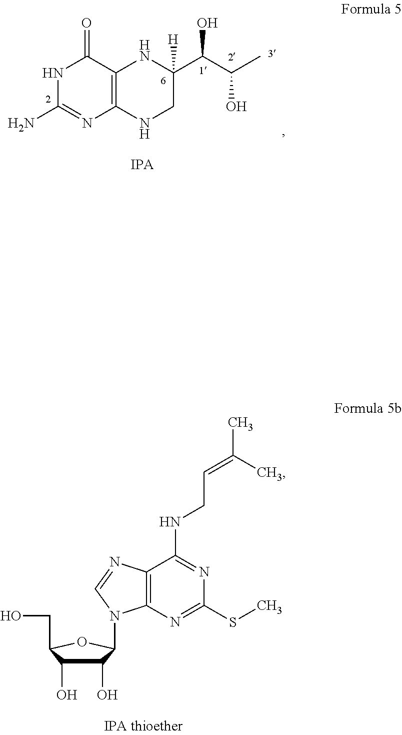

6. The substance of claim 5 administered as an oral, sublingual, injectable (intravenous, intratumor, subcutaneous, intraperitoneal, intracranial or intrathecal), inhalable, aerosolized, respirable, topical (including ophthalmic, otic), transdermal, or suppository in amounts sufficient to maintain normal levels of isopentenylated tRNA during EGSS performed according to the method of claim 1, in a mammal in need of such treatment.

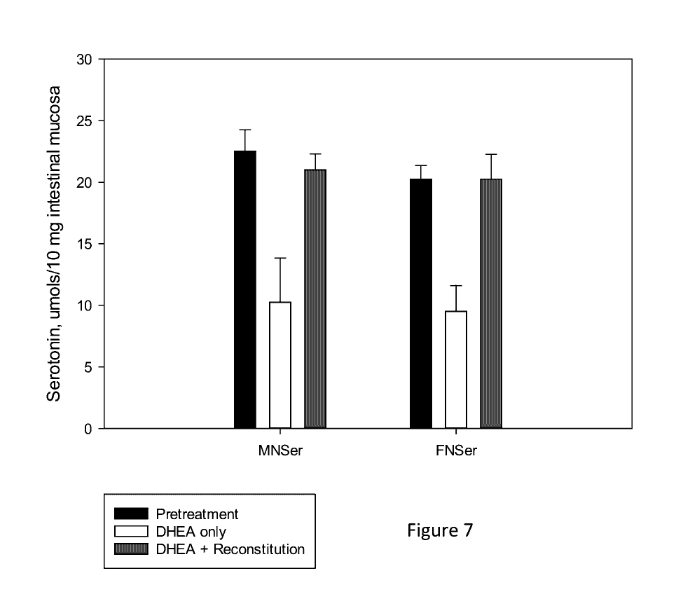

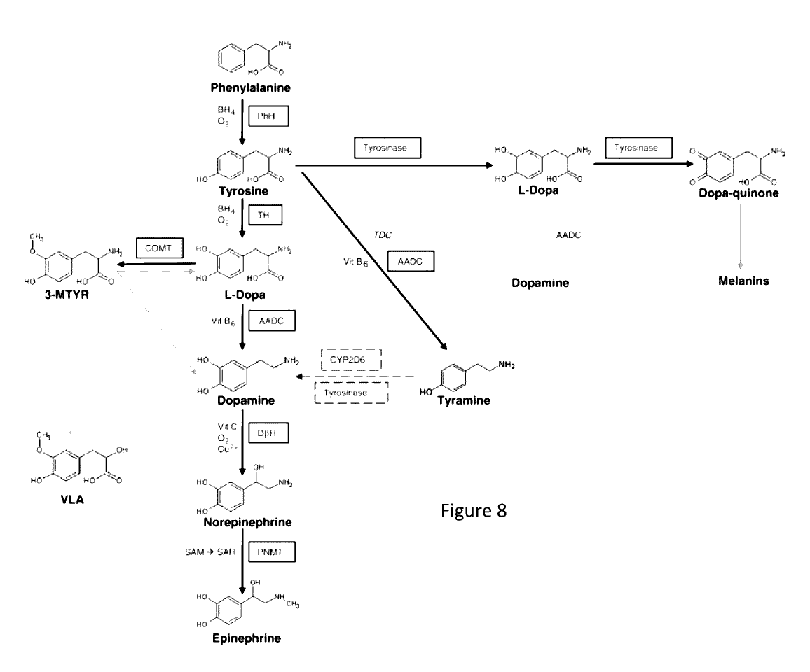

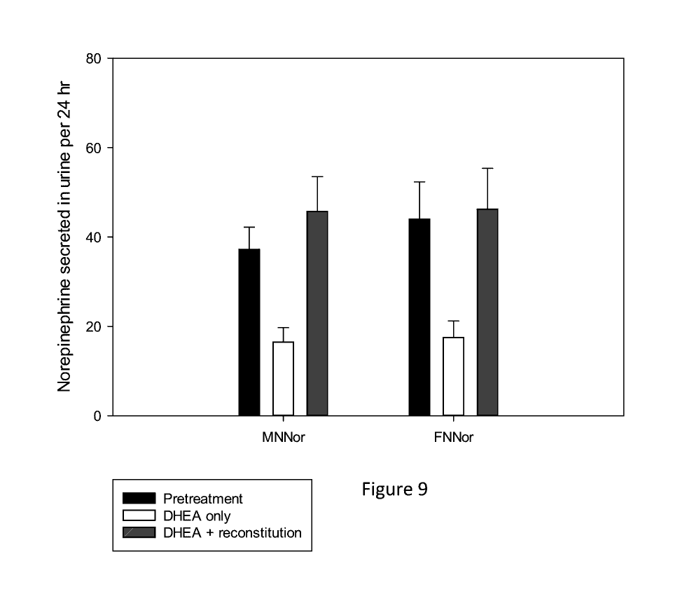

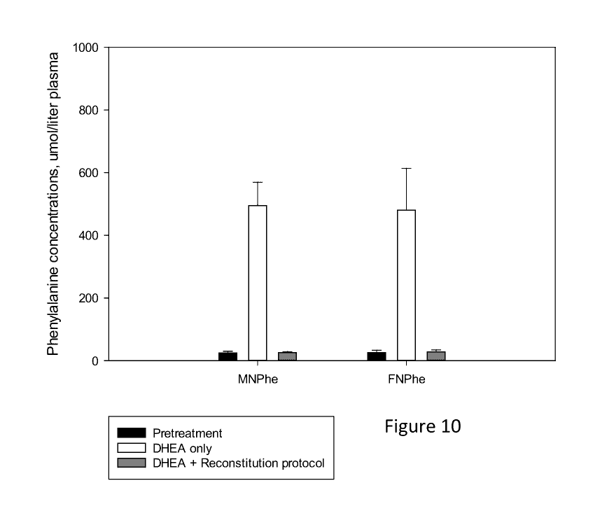

7. The method of claim 1, comprising administration of a nitric oxide donor, e.g., potassium nitrate (KNO3, FIG. 7) or other nitric oxide donor, e.g., sodium nitrite (NaNO2, Formula 7b) as a component of the reconstitution mixture co-administered with the substance(s) of claim 1, in amounts sufficient to maintain nitric oxide-dependent physiological systems otherwise impaired during DHEA-mediated EGSS.

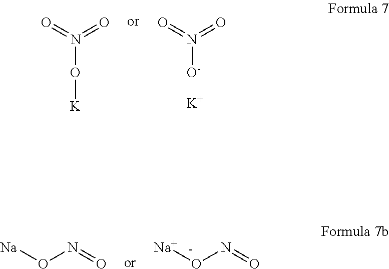

8. The substance of claim 7 administered as an oral, sublingual, injectable (intravenous, intratumor, subcutaneous, intraperitoneal, intracranial or intrathecal), inhalable, aerosolized, respirable, topical (including ophthalmic, otic), transdermal, or suppository in amounts sufficient to prevent side effects of nitric oxide depletion during EGSS performed according to the method of claim 1, in a mammal in need of such treatment.

9. The method of claim 1, comprising administration of α, β, γ and/or δ Tocotrienol(s) (Formula 9) as a component of the reconstitution mixture co-administered with the substances of claim 1, in amounts sufficient to maintain or contribute to the maintenance of healthy cellular ubiquinone levels during DHEA-mediated EGSS.

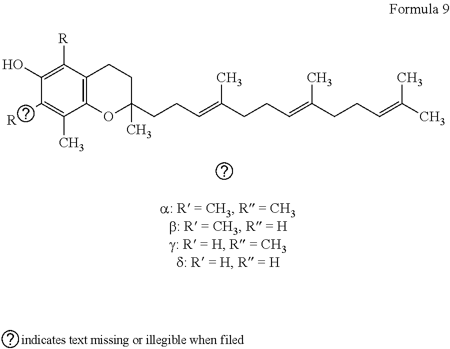

10. The substance of claim 9 administered as an oral, sublingual, injectable (intravenous, intratumor, subcutaneous, intraperitoneal, intracranial or intrathecal), inhalable, aerosolized, respirable, topical (including ophthalmic, otic), transdermal, or suppository in amounts sufficient to reduce ubiquinone depletion during EGSS performed according to the method of claim 1, in a mammal in need of such treatment.

11. The method of claim 1, comprising administration of ubiquinone (formula 11, chain length 6-10) or ubiquinol (formula 11b, chain length 6-10) as a component of the reconstitution mixture co-administered with the substances of claim 1, ubiquinone administered in sufficient amounts to maintain healthy cellular ubiquinone levels during DHEA-mediated EGSS.

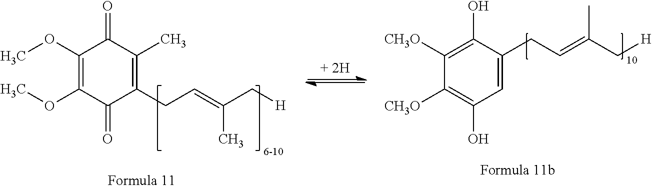

12. The substance of claim 11 administered as an oral, sublingual, injectable (intravenous, intratumor, subcutaneous, intraperitoneal, intracranial or intrathecal), inhalable, aerosolized, respirable, topical (including ophthalmic, otic), transdermal, or suppository in amounts sufficient to maintain adequate ubiquinone levels during EGSS performed according to the method of claim 1, in a mammal in need of such treatment.

13. The method of claim 1, comprising administration of folinic acid (formula 13) as a component of the reconstitution mixture co-administered with the substance(s) of claim 1, in sufficient amounts to provide adequate amounts of folate pool products during DHEA-mediated EGSS.

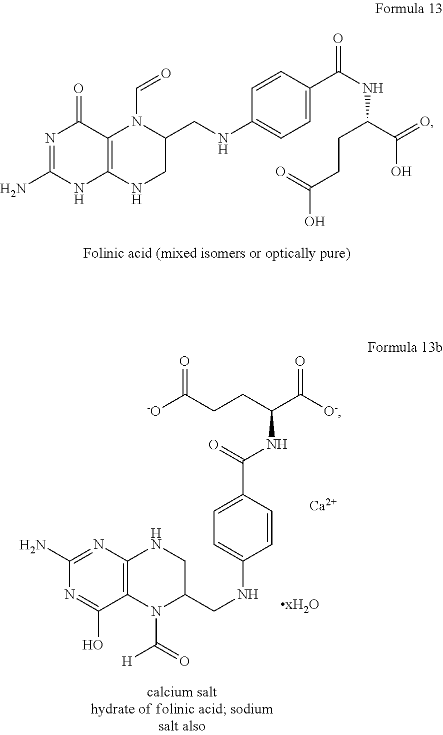

14. The substance of claim 13 administered as an oral, sublingual, injectable (intravenous, intratumor, subcutaneous, intraperitoneal, intracranial or intrathecal), inhalable, aerosolized, respirable, topical (including ophthalmic, otic), transdermal, or suppository in amounts sufficient to maintain adequate levels of folate pathway products during EGSS performed according to the method of claim 1, in a mammal in need of such treatment.

15. The method of claim 1, comprising administration of 5-Hydroxtryptophan (5-HT; formula 14a), pyridoxine (formula 14b), L-DOPA (formula 14c), S-adenosylmethionine (SAMe; Formula 14d), ascorbate (ascorbic acid; formula 14e), pantothenic acid (formula 14f), and zinc as components of the reconstitution mixture co administered with the substance(s) of claim 1, in sufficient amounts to maintain healthy levels of monamines.

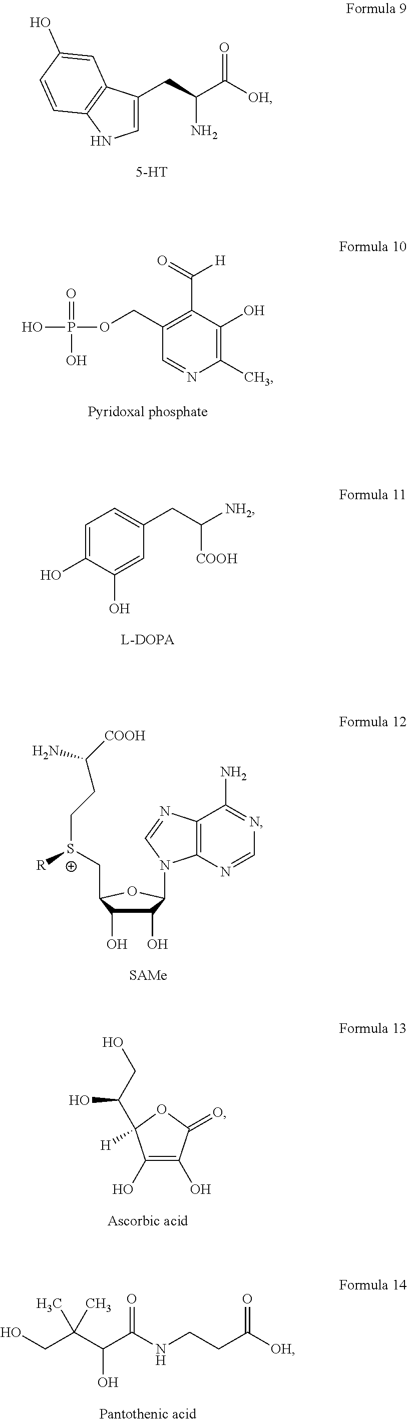

16. The substances of claim 15 administered as oral, sublingual, injectable (intravenous, intratumor, subcutaneous, intraperitoneal, intracranial or intrathecal), inhalable, aerosolized, respirable, topical (including ophthalmic, otic), transdermal, or suppository in amounts sufficient to maintain adequate monoamine leels during EGSS performed according to the method of claim 1, in a mammal in need of such treatment.

17. A method for restoring normal or near normal steroid hormone levels in neutered animals with DHEA, without inducing auto-inflammatory and other side effects of such treatment, comprising administration of the substance(s) of claim 1, the substance(s) of claim 3, the substance(s) of claim 5, the substance(s) of claim 7, the substances of claim 9, the substances of claim 11, the substances of claim 13, and the substances of claim 15, administered in any combination and at any time that reduces or eliminates the negative side effects of EGSS performed according to the method of claim 1.

18. The method of claim 13 in which folinic acid is substituted for by adenine, its nucleosides or nucleotides, or hypoxanthine, and uracil, its nucleosides and nucleotides.

19. The method of claim 13 in which adenosine, adenosine monophosphate, adenosine diphosphate or adenosine triphosphate are administered in addition to folinic acid to remediate adenosine depletion at local sites.

20. The method of claim 5 in which IPA is substituted for by mevalonic acid, its salts, hydrates, and saponates, or by the isoprenoid donors geranylgeraniol, geraniol, or farnesol.
